(19) United States
(12) Patent Application Publication
(10) Pub. No.: US 2026/0055471 A1
(43) Pub. Date: Feb. 26, 2026

(54) KITS AND METHODS USEFUL FOR PROGNOSING, DIAGNOSING, AND TREATING PROSTATE CANCER

(71) Applicants: The Regents of the University of Michigan, Ann Arbor, MI (US); The Regents of the University of California, Oakland, CA (US)

(72) Inventors: Ana Maria Kenney, Irvine, CA (US); Bin Yu, Berkeley, CA (US); Tiffany Tang, Ann Arbor, MI (US); Yuping Zhang, Ann Arbor, MI (US); Arul M. Chinnaiyan, Ann Arbor, MI (US)

(21) Appl. No.: 19/308,839

(22) Filed: Aug. 25, 2025

Related U.S. Application Data
(63) Continuation of application No. PCT/US2025/042703, filed on Aug. 20, 2025.
(60) Provisional application No. 63/685,330, filed on Aug. 21, 2024.

(51) Int. Cl. C12Q 1/6886; C12N 15/10

(52) U.S. Cl. CPC C12Q 1/6886; C12N 15/1096; C12Q 2600/106; C12Q 2600/112; C12Q 2600/118; C12Q 2600/158

(57) ABSTRACT

Provided herein are kits and methods useful for cancer diagnosis, prognosis, research and therapy. In particular, provided herein are methods of diagnosing, prognosing, and/or treating prostate cancer based on expression levels of cancer markers.

### STATEMENT OF RELATED APPLICATIONS

This application is a continuation of PCT International Application No. PCT/US2025/042703, filed Aug. 20, 2025, which claims priority to and the benefit of U.S. Provisional Patent Application No. 63/685,330, filed Aug. 21, 2024, the entire contents of which are incorporated herein by reference for all purposes.

### STATEMENT REGARDING FEDERALLY SPONSORED RESEARCH OR DEVELOPMENT

This invention was made with government support under CA186786, CA271854, and CA231996 awarded by the National Institutes of Health. The government has certain rights in the invention.

### SEQUENCE LISTING

The text of the computer readable sequence listing filed herewith, titled “UM-43302-302_SQL.xml”, created Aug. 25, 2025, having a file size of 18,682 bytes, is hereby incorporated by reference in its entirety.

### FIELD OF THE DISCLOSURE

Provided herein are kits and methods useful for cancer diagnosis, prognosis, research and therapy. In particular, provided herein are methods of diagnosing, prognosing, and/or treating prostate cancer based on expression levels of cancer markers.

### BACKGROUND OF THE DISCLOSURE

Prostate cancer is the third most common urologic malignancy and can originate from the prostate parenchyma or urinary collecting system. Prostate cell carcinoma, arising from the prostate parenchyma, is the most common malignant prostate tumor associated with an incidence of 64,000 cases and approximately 14,000 deaths yearly in the United States. From the urinary collecting system, urothelial cell carcinoma is the most common malignancy representing approximately 10-15% of all prostate tumors. The overall incidence of malignant prostate tumors is increasing and currently is the third most common form of genitourinary cancer. Both malignant and benign prostate tumors are increasingly diagnosed in incidental fashion with the use of advanced cross-sectional imaging. Accurate diagnosis of benign versus malignant tumor types is lacking and accordingly patients might be subjected to unnecessary treatment or overtreatment. Furthermore, there are currently no diagnostic tests from needle biopsy, urine or blood that accurately characterize prostate tumors or identify patients at risk for prostate tumors. The diagnostic and therapeutic approach to prostate tumors is complicated by the presence of multiple benign prostate tumor types and the fact that many small malignant prostate parenchymal tumors can be observed rather than definitively treated.

Early detection and treatment of aggressive prostate cancers are critical to reducing its harms, but current diagnostic tests are unable to reliably identify clinically significant (e.g., classified as Grade Group [GG]≥2) prostate cancer. Poorly specific for cancer, the harms of serum prostate-specific antigen (PSA) as an isolated diagnostic tool are well-documented, and several cancer-specific biomarkers have been proposed to augment PSA. These tools have demonstrated incremental benefit, potentially avoiding 15-30% of biopsies performed due to PSA, at the cost of failing to diagnose 8-15% of GG≥2 prostate cancer. MRI has been similarly used in this role at several academic centers. In addition to mounting evidence that a proportion of GG≥2 cancers are MRI-invisible, MRI is costly, resource-intensive, and subjectively interpreted, making it less practical as a population-level diagnostic tool. Thus, there continues to be a critical need for a practical (affordable, reproducible, standardizable) non-invasive test to reliably detect aggressive prostate cancer in a localized, curable state.

While several molecular mechanisms yield aggressive prostate cancer biology, most patients harbor tumors reflecting a limited number of these mechanisms. At the present time, there are no accurate, user-friendly, and widely accessible screening tools at the tissue, blood or urinary level for ideal clinical management of prostate tumors.

### SUMMARY OF THE DISCLOSURE

Provided herein are methods of treating prostate cancer, comprising: a) assaying the level of expression of TMPRSS2-ERG, SCHLAP1, OR51E2, PCAT14, PCA3, and KLK4 and optionally APOC1 in a sample from a subject diagnosed with prostate cancer; and b) administering a prostate cancer treatment to a subject identified as having altered levels of expression of the genes relative to a subject without prostate cancer or a subject with low-grade prostate cancer.

Further provided are methods of characterizing, prognosing, or recommending a treatment for prostate cancer, comprising: a) assaying the level of expression of TMPRSS2-ERG, SCHLAP1, OR51E2, PCAT14, PCA3, and KLK4 and optionally APOC1 in a sample from a subject diagnosed with prostate cancer; and b) identifying said subject as having high-grade prostate cancer when the subject is identified as having altered levels of expression of the genes relative to a subject without prostate cancer or a subject with low-grade prostate cancer.

Further provided are methods for informing a prostate cancer survival outcome, comprising: (i) detecting an amount of expression of TMPRSS2-ERG, SCHLAP1, OR51E2, PCAT14, PCA3, and KLK4 and optionally APOC1, wherein the amount of expression is present in urine from a subject; (ii) determining a score based on the amount of expression, wherein the score correlates with or informs the subject's likelihood of having or developing Grade Group ≥2 prostate cancer; and (iii) generating a report comprising the score.

Further provided are methods for identifying a subject having a high likelihood of having or developing Grade Group ≥2 prostate cancer, comprising detecting an amount of expression of TMPRSS2-ERG, SCHLAP1, OR51E2, PCAT14, PCA3, and KLK4 and optionally APOC1, wherein the amount of expression is present in the subject's urine and indicates with a diagnostic accuracy (AUC) of ≥0.75 (e.g., greater than 0.78) whether the subject has a high likelihood of having a Grade Group ≥2 prostate cancer.

Further provided are methods for identifying a likelihood of detecting Grade Group ≥2 prostate cancer from a prostate biopsy of a subject, the method comprising detecting an amount of expression of TMPRSS2-ERG, SCHLAP1, OR51E2, PCAT14, PCA3, and KLK4 and optionally APOC1, wherein the amount of expression is present in the subject's urine and indicates with a diagnostic accuracy (AUC) of ≥0.75 (e.g., greater than 0.78) the likelihood that Grade Group ≥2 prostate cancer would be detected from the prostate biopsy of the subject.

Further provided are methods for screening for an amount of expression of TMPRSS2-ERG, SCHLAP1, OR51E2, PCAT14, PCA3, and KLK4 and optionally APOC1 genes, comprising: (a) allowing a sample of urine from a human subject to react with a reagent for detecting an amount of expression of TMPRSS2-ERG, SCHLAP1, OR51E2, PCAT14, PCA3, and KLK4 and optionally APOC1; and (b) detecting the amount of expression of the genes, wherein the amount of expression is present in the sample and the detecting comprises using an in vitro assay.

Further provided are methods for detecting an amount of mRNA expressed by TMPRSS2-ERG, SCHLAP1, OR51E2, PCAT14, PCA3, and KLK4 and optionally APOC1, comprising: (a) synthesizing cDNA from mRNA that is expressed by TMPRSS2-ERG, SCHLAP1, OR51E2, PCAT14, PCA3, and KLK4 and optionally APOC1; (b) amplifying the cDNA to provide amplified cDNA; and (c) detecting the amplified cDNA, wherein the amplified cDNA indicates the amount of mRNA expressed by the genes.

Further provided are methods for detecting an amount of mRNA expressed by TMPRSS2-ERG, SCHLAP1, OR51E2, PCAT14, PCA3, and KLK4 and optionally APOC1, comprising: (a) isolating nucleic acid from a first composition comprising urine from a human subject to provide isolated nucleic acid; (b) allowing the isolated nucleic acid to react with a second composition comprising a reagent for detecting the amount of mRNA that is present in the first composition and expressed by TMPRSS2-ERG, SCHLAP1, OR51E2, PCAT14, PCA3, and KLK4 and optionally APOC1; and (c) detecting the amount of mRNA expressed by the genes.

Further provided are kits comprising: a container, the container containing a reagent composition for detecting an amount of expression of TMPRSS2-ERG, SCHLAP1, OR51E2, PCAT14, PCA3, and KLK4 and optionally APOC1; and instructions for detecting the amount of expression, where the amount of expression is present in a subject's urine.

Additional embodiments are described herein.

### DESCRIPTION OF THE FIGURES

FIG. 1 shows an overview of PCS-guided model development and validation pipeline for s7MPS2.

FIGS. 2A-2B shows cross-validation AUROC across various prediction models and data preprocessing

pipelines in prediction check stage. (FIG. 2A) For each choice of data preprocessing and prediction model, the validation AUROC, averaged across 4 CV folds and 10 repeated Development-Test splits, is shown.

(FIG. 2B) Comparison of the variation in AUROC across data preprocessing pipelines (left) and methods (right).

FIGS. 3A-3F shows importance and stability of top genes across data preprocessing and modeling choices. Top 15 ranked genes are summarized according to (FIG. 3A) their mean gene ranking across four

data preprocessing pipelines and six prediction-checked models, alongside (FIG. 3B) the variability of their gene rankings as measured by the standard deviation (SD) of this distribution and (FIGS. 3C-3E) the proportion of times that the gene appeared in the top 5, 10, and 17 genes. (FIG. 3F) The heatmap shows a more granular view of the gene rankings, displaying (in text and color) the mean gene ranking per data preprocessing and model choice, averaged across 10 Development-Test splits.

FIG. 4 shows a test of AUROC from logistic ridge regression across different choices of gene panel sizes, gene rankings, and data preprocessing pipelines.

FIG. 5 shows blinded external validation AUROC curves for s7MPS2/s8MPS2 and comparison models. The AUROC curves from the blinded external validation cohort are shown for various urine-based biomarker tests without prostate volume (MPS, MPS2, s7MPS2, and s8MPS2, left) and with prostate volume (MPS2+, s7MPS2+, and s8MPS2+ right).

FIG. 6 shows an overview of data splitting scheme for developing simplified MPS2 models. Lines 3-5 correspond to the prediction check stage. Line 6 corresponds to the stability-driven gene ranking stage. Lines 7-10 correspond to the internal validation stage.

FIG. 7 is a schematic block diagram of an example system that includes a compute device that can be used to implement methods or steps described herein, according to an embodiment.

### DEFINITIONS

To facilitate an understanding of the present disclosure, a number of terms and phrases are defined below:

As used herein, the terms “detect”, “detecting” or “detection” may describe either the general act of discovering or discerning or the specific observation of a composition. Detecting a composition may comprise determining the presence or absence of a composition. Detecting may comprise quantifying a composition. For example, detecting comprises determining the expression level of a composition. The composition may comprise a nucleic acid molecule. For example, the composition may comprise at least a portion of the cancer markers disclosed herein. Alternatively, or additionally, the composition may be a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans. In some embodiments, the subject is a mammal having a prostate. In some embodiments, the subject is a human having a prostate.

The term “diagnosed,” as used herein, refers to the recognition of a disease by its signs and symptoms, or genetic analysis, pathological analysis, histological analysis, and the like.

As used herein, the language “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of cancer marker genes, including but not limited to, the cancer markers disclosed herein.

As used herein, the language “stage of cancer” refers to a qualitative or quantitative assessment of the level of advancement of a cancer. Criteria useful to determine the stage of a cancer include, but are not limited to, the size of the tumor and the extent of metastases (e.g., localized or distant).

As used herein, the term “high likelihood” when used, for example, in reference to the likelihood of having or developing prostate cancer (e.g., Grade Group ≥2 prostate cancer) refers to an increased likelihood of developing Grade Group ≥2 prostate cancer relative to a low-risk subject or a high absolute likelihood of developing Grade Group ≥2 prostate cancer. In some embodiments, a high likelihood of developing Grade Group ≥2 prostate cancer is determined based on the level of expression of 1 or more genes described herein. In some embodiments, a “high likelihood” is a likelihood that is increased by 50%, 100%, 200%, 500%, or more relative to a healthy subject or a subject that does not have altered expression of genes recited herein. In some embodiments, a high likelihood refers to the absolute likelihood of developing Grade Group ≥2 prostate cancer. In some embodiments, a “high likelihood” is a 50% or greater likelihood that a subject's prostate biopsy would detect Grade Group ≥2 prostate cancer in the subject. In some embodiments, a “high likelihood” is a 60% or greater likelihood that a subject's prostate biopsy would detect Grade Group ≥2 prostate cancer in the subject. In some embodiments, a “high likelihood” is a 70% or greater likelihood that a subject's prostate biopsy would detect Grade Group ≥2 prostate cancer in the subject. In some embodiments, a “high likelihood” is a 80% or greater likelihood that a subject's prostate biopsy would detect Grade Group ≥2 prostate cancer in the subject. In some embodiments, a “high likelihood” is a 90% or greater likelihood that a subject's prostate biopsy would detect Grade Group ≥2 prostate cancer in the subject. In some embodiments, a “high likelihood” is a 95% or greater likelihood that a subject's prostate biopsy would detect Grade Group ≥2 prostate cancer in the subject. In some embodiments, a “high likelihood” is a 96% or greater likelihood that a subject's prostate biopsy would detect Grade Group ≥2 prostate cancer in the subject. In some embodiments, a “high likelihood” is a 97% or greater likelihood that a subject's prostate biopsy would detect Grade Group ≥2 prostate cancer in the subject. In some embodiments, a “high likelihood” is a 98% or greater likelihood that a subject's prostate biopsy would detect Grade Group ≥2 prostate cancer in the subject. In some embodiments, a “high likelihood” is a 99% or greater likelihood that a subject's prostate biopsy would detect Grade Group ≥2 prostate cancer in the subject. In some embodiments, a “high likelihood” is a 100% likelihood that a subject's prostate biopsy would detect Grade Group ≥2 prostate cancer in the subject.

As used herein, the language “low likelihood” when used, for example, in reference to the likelihood of having or developing prostate cancer (e.g., Grade Group ≥2 prostate cancer) refers to a decreased likelihood of developing prostate cancer (e.g., Grade Group ≥2 prostate cancer) relative to an average-risk subject or a low absolute likelihood of developing prostate cancer (e.g., Grade Group ≥2 prostate cancer). In some embodiments, a low likelihood of developing Grade Group ≥2 prostate cancer is determined based on the level of expression of 1 or more genes described herein. In some embodiments, a “low likelihood” is a likelihood that is decreased by 50%, 100%, 200%, 500%, or more relative to a healthy subject or a subject that does not have altered expression of genes recited herein. In some embodiments, a low likelihood refers to the absolute likelihood of developing Grade Group ≥2 prostate cancer. In some embodiments, a “low likelihood” is a less than 50% likelihood that a subject's prostate biopsy would detect Grade Group ≥2 prostate cancer in the subject. In some embodiments, a “low likelihood” is a 40% or less likelihood that a subject's prostate biopsy would detect Grade Group ≥2 prostate cancer in the subject. In some embodiments, a “low likelihood” is a 30% or less likelihood that a subject's prostate biopsy would detect Grade Group ≥2 prostate cancer in the subject. In some embodiments, a “low likelihood” is an 20% or less likelihood that a subject's prostate biopsy would detect Grade Group ≥2 prostate cancer in the subject. In some embodiments, a “low likelihood” is a 10% or less likelihood that a subject's prostate biopsy would detect Grade Group ≥2 prostate cancer in the subject. In some embodiments, a “low likelihood” is a 5% or less likelihood that a subject's prostate biopsy would detect Grade Group ≥2 prostate cancer in the subject. In some embodiments, a “low likelihood” is a 4% or less likelihood that a subject's prostate biopsy would detect Grade Group ≥2 prostate cancer in the subject. In some embodiments, a “low likelihood” is a 3% or less likelihood that a subject's prostate biopsy would detect Grade Group ≥2 prostate cancer in the subject. In some embodiments, a “low likelihood” is a 2% or less likelihood that a subject's prostate biopsy would detect Grade Group ≥2 prostate cancer in the subject. In some embodiments, a “low likelihood” is a 1% or less likelihood that a subject's prostate biopsy would detect Grade Group ≥2 prostate cancer in the subject. In some embodiments, a “low likelihood” is a 0% likelihood that a subject's prostate biopsy would detect Grade Group ≥2 prostate cancer in the subject.

As used herein, the language “nucleic acid molecule” refers to any nucleic acid containing molecule, including but not limited to, DNA or RNA. The nucleic acid molecule may comprise one or more nucleotides. The language may include nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. The term further encompasses sequences that may include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladeninc, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladeninc, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. It will be understood that when a nucleotide sequence is represented by a DNA sequence (i.e., A, T, G, C), the sequence also includes an RNA sequence (i.e., A, U, G, C) in which “U” replaces “T.”

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both 5′ and 3′ ends for a distance of about 1 kb or more on either end, such that the “gene” corresponds to the length of the full-length mRNA. Sequences located 5′ of the coding region and present on the mRNA are referred to as 5′ non-translated or untranslated sequences. Sequences located 3′ or downstream of the coding region and present on the mRNA are referred to as 3′ non-translated or untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 nucleotide residues long (e.g., between 15 and 100), however, as used herein, the term is also intended to encompass longer polynucleotide chains. Oligonucleotides are often referred to by their length. For example, a 24-residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

The term “label” as used herein refers to any atom or molecule that can be used to provide a detectable (preferably quantifiable) effect, and that can be attached to a nucleic acid or protein. Labels include but are not limited to: dyes; radiolabels such as ³²P; binding moieties such as biotin; haptens such as digoxgenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (e.g., a positive or negative charge) or alternatively, may be charge neutral. Labels can include or consist of nucleic acid or protein sequence, so long as the sequence comprising the label is detectable. In some embodiments, nucleic acids are detected directly without a label (e.g., directly reading a sequence).

As used herein, the term “sample” includes a specimen or culture obtained from any source, as well as biological and environmental samples. Biological samples may be obtained from animals (including humans) and encompass fluids (e.g., blood, urine), solids, tissues, and gases. Biological samples can include urine, urine supernatant, and urine cell pellet as well as blood products, such as plasma, serum and the like. Such examples are not however to be construed as limiting the sample types applicable to the present disclosure.

As used herein, “high-grade prostate cancer” means Grade Group ≥2 prostate cancer. In some embodiments, the high-grade prostate cancer is GG≥3 prostate cancer.

As used herein, “low-grade prostate cancer” means Grade Group <2 prostate cancer.

As used herein, “score” is a likelihood that a subject's prostate biopsy would detect Grade Group ≥2 prostate cancer in the subject, i.e., that a subject's prostate biopsy would be positive for a prostate cancer. The score is based on the level or amount of expression of genes described herein present in a sample from a subject. In some embodiments, the score is a numerical value ranging from 0% to 100%. In some embodiments, the numerical value is expressed as a decimal number ranging from 0.0 to 100.0. In some embodiments, the score is a qualitative read-out of “low risk” or “elevated risk”.

As used herein, the term “altered,” for example in the context of “altered levels of expression of one or more of the genes,” refers to a level of gene expression that is different (e.g., increased or decreased) than the level of expression in, e.g., a subject without prostate cancer or a subject with low-grade prostate cancer.

As used herein, the term “variant,” e.g., a gene variant, refers to a sequence change that does not affect gene identity. Such sequence changes are readily appreciated by the skilled artisan. In some embodiments, a variant comprises a mutation, a substitution, and/or a deletion. In some embodiments, a variant comprises a polymorphism. In some embodiments, a variant comprises a splice variant.

As used herein, the term “about” means±10% variation from nominal value unless otherwise indicated or inferred. When the term “about” is used before a number, the present disclosure also includes the specific number itself, unless specifically stated otherwise.

### DETAILED DESCRIPTION OF THE DISCLOSURE

Provided herein are kits and methods useful for cancer diagnosis, prognosis, research and therapy. In particular, provided herein are methods of diagnosing, prognosing, and/or treating prostate cancer based on expression levels of cancer markers.

The disclosure is based, at least in part, on the discovery of methods for determining a likelihood that a subject has Grade Group ≥2 prostate cancer based on an amount of expression of genes described herein.

Described herein are methods and kits incorporating markers useful for prognosing, diagnosing or treating prostate cancer. Importantly, detection of PSA (prostate specific antigen), the conventional method for prognosis and/or diagnosis of prostate cancer, is not a necessary step of the methods described herein. PSA elevation identified during PSA screening leads to a high rate of invasive and unnecessary biopsies in men without cancer and frequent overdiagnosis of low-grade, indolent cancers (grade group 1 (GG1)). The kits and methods of the present disclosure provide more precise prognosis or diagnosis of prostate cancer and help identify those subjects that can benefit from early, aggressive therapeutic interventions while sparing those subjects with indolent disease from an invasive procedure, such as a biopsy. The instant methods therefore provide a set of prostate cancer biomarkers, and particularly high-grade (e.g., GG≥2) prostate cancer biomarkers, independent of PSA.

Accordingly, provided herein are methods and kits useful for prognosing, diagnosing or treating subjects with prostate cancer, in some embodiments, Grade Group ≥2 prostate cancer. For example, in some embodiments, provided herein are methods of treating prostate cancer, comprising: a) assaying a level of expression of TMPRSS2-ERG, SCHLAP1, OR51E2, PCAT14, PCA3, and KLK4 and optionally APOC1 in a sample from a subject prognosed or diagnosed with prostate cancer; and b) administering a prostate cancer treatment to a subject identified as having altered levels of expression of the genes relative to a subject without prostate cancer or a subject with low-grade prostate cancer. In some embodiments, the subject has high-grade prostate cancer.

Further embodiments provide methods of characterizing, prognosing, or recommending a treatment for prostate cancer, comprising: a) assaying a level of expression of TMPRSS2-ERG, SCHLAP1, OR51E2, PCAT14, PCA3, and KLK4 and optionally APOC1 in a sample from a subject prognosed or diagnosed with prostate cancer; and b) identifying said subject as having high-grade prostate cancer when the subject is identified as having altered levels of expression of the genes relative to a subject without prostate cancer or a subject with low-grade prostate cancer. In some embodiments, the methods further comprise administering a prostate cancer treatment to the subject. In some embodiments, the methods further comprise administering a treatment for Grade Group ≥2 prostate cancer to the subject.

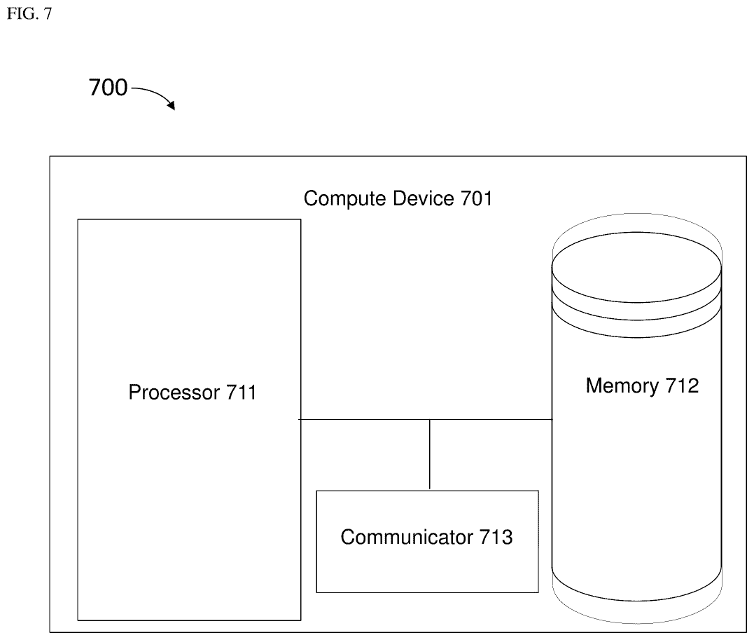

In some embodiments, the methods further comprise performing a prostate biopsy on the subject. In some embodiments, the methods further comprise recommending to the subject or the subject's health care provider (e.g., via a compute device, such as compute device 701 of FIG. 7, used or accessible by the subject or the subject's healthcare provider) that the subject undergo a prostate biopsy. In some embodiments, the prostate biopsy indicates the subject has Grade Group ≥2 prostate cancer. In some embodiments, the prostate biopsy indicates the subject does not have Grade Group ≥2 prostate cancer.

In some embodiments, the methods further comprise recommending to the subject or the subject's health care provider that the subject does not undergo a prostate biopsy.

In some embodiments, the methods do not comprise performing a prostate biopsy on the subject.

The methods described herein are useful to identify subjects with high-grade prostate cancer for treatment and allow those identified as not having high-grade prostate cancer to avoid a biopsy or treatment and, accordingly, its associated side effects. The methods as provided herein are useful to reduce the number of unnecessary prostate biopsies, sparing healthy subjects from a costly, invasive procedure.

### I. Methods of Assaying Marker Expression

As described herein, embodiments of the present disclosure provide methods for prognosis, diagnosis or treatment that utilize detection of an expression amount or level of TMPRSS2-ERG, SCHLAP1, OR51E2, PCAT14, PCA3, and KLK4 and optionally APOC1. Illustrative, non-limiting methods are described herein.

### Genes for Detecting

In some embodiments, the level or amount of expression of the genes described herein is determined. In some embodiments, the level or amount of expression is the level or amount of mRNA or protein expressed by the genes.

In some embodiments, the genes are TMPRSS2-ERG, SCHLAP1, OR51E2, PCAT14, PCA3, and KLK4 and optionally APOC1. Details of each of the genes are described below.

In some embodiments, the methods and kits described herein are useful for detecting a level or an amount of expression of a TMPRSS2-ERG gene. A TMPRSS2-ERG gene fusion overexpresses the transcription factor ERG, which is present in both early- and late-stage prostate cancer. Numerous variations of TMPRSS2-ERG fusions have been identified, with the most common comprising exon 1 of TMPRSS2 and exons 4-11 of ERG. In some embodiments, a TMPRSS2-ERG gene fusion comprises a fusion of the nucleotide sequences of Ensembl gene identifiers ENSG00000184012 and ENSG00000157554. In some embodiments, a TMPRSS2-ERG gene fusion comprises the nucleotide sequence of SEQ ID NO: 1 or a variant thereof.

In some embodiments, the methods and kits described herein are useful for detecting a level or an amount of expression of a SCHLAP1 gene. SCHLAP1 is a long noncoding RNA overexpressed in a subset of prostate cancers. SCHLAP1 antagonizes the genome-wide localization and regulatory functions of the SWI/SNF chromatin-modifying complex. In some embodiments, the SCHLAP1 gene comprises the nucleotide sequence provided by the HUGO Gene Nomenclature Committee (HGNC). In some embodiments, the HGNC identifier for SCHLAP1 is 48603. In some embodiments, the SCHLAP1 gene is located at chromosome position 2q31.3. In some embodiments, a SCHLAP1 gene comprises the nucleotide sequence of Ensembl gene identifier ENSG00000281131. In some embodiments, a SCHLAP1 gene comprises the nucleotide sequence of SEQ ID NO:2 or a variant thereof.

In some embodiments, the methods and kits described herein are useful for detecting a level or an amount of expression of a OR51E2 gene. OR51E2 is an odorant receptor (OR) which represent the largest G protein-coupled receptor (GPCR) family in the human genome. Activation of human ORs can influence cell proliferation. Specifically, OR51E2 has been identified as being involved in the regulation of cell growth, migration and the invasiveness of melanocytes, melanoma cells, and prostate cancer cells. In some embodiments, the OR51E2 gene comprises the nucleotide sequence provided by HGNC. In some embodiments, the HGNC identifier for OR51E2 is 15195. In some embodiments, the OR51E2 gene is located at chromosome position 11p15.4. In some embodiments, an OR51E2 gene comprises the nucleotide sequence of Ensembl gene identifier ENSG00000167332. In some embodiments, an OR51E2 gene comprises the nucleotide sequence of SEQ ID NO:3 or a variant thereof.

In some embodiments, the methods and kits described herein are useful for detecting a level or an amount of expression of an APOC1 gene. APOC1 is the smallest apolipoprotein and is a component of both triglyceride-rich lipoproteins and high-density lipoproteins. APOC1 is involved in various biological processes and is related to the progression of multiple diseases such as diabetic nephropathy, Alzheimer's disease, and glomerculosclerosis. Recent studies have shown APOC1 may be associated with the development of cancers, including breast cancer, pancreatic cancer, lung cancer, and prostate cancer. In some embodiments, the APOC1 gene comprises the nucleotide sequence provided by HGNC. In some embodiments, the HGNC identifier for APOC1 is 607. In some embodiments, the APOC1 gene is located at chromosome position 19q13.32. In some embodiments, an APOC1 gene comprises the nucleotide sequence of Ensembl gene identifier ENSG00000130208. In some embodiments, an APOC1 gene comprises the nucleotide sequence of SEQ ID NO:4 or a variant thereof.

In some embodiments, the methods and kits described herein are useful for detecting a level or an amount of expression of a PCAT14 gene. PCAT14 is a long non-coding RNA that exhibits both cancer and lineage specificity. PCAT14 is transcriptionally regulated by androgen receptor (AR) and endogenous PCAT14 overexpression suppresses cell invasion. In some embodiments, the PCAT14 gene comprises the nucleotide sequence provided by HGNC. In some embodiments, the HGNC identifier for PCAT14 is 48977. In some embodiments, the PCAT14 gene is located at chromosome position 22q11.23. In some embodiments, a PCAT14 gene comprises the nucleotide sequence of Ensembl gene identifier ENSG00000280623. In some embodiments, a PCAT14 gene comprises the nucleotide sequence of SEQ ID NO:5 or a variant thereof.

In some embodiments, the methods and kits described herein are useful for detecting a level or an amount of expression of a PCA3 gene. PCA3 is a non-coding gene associated with prostate cancer. In some embodiments, the PCA3 gene comprises the nucleotide sequence provided by HGNC. In some embodiments, the HGNC identifier for PCA3 is 8637. In some embodiments, the PCA3 gene is located at chromosome position 9921.2. In some embodiments, a PCA3 gene comprises the nucleotide sequence of Ensembl gene identifier ENSG00000225937. In some embodiments, a PCA3 gene comprises the nucleotide sequence of SEQ ID NO:6 or a variant thereof.

In some embodiments, the methods and kits described herein are useful for detecting a level or an amount of expression of a KLK4 gene. KLK4 is a member of the kallikrein (KLK) family of highly conserved serine proteases that play key roles in a variety of physiological and pathological processes. KLKs are secreted proteins that have extracellular substrates and function. KLK4 is overexpressed in prostate cancer. In some embodiments, the KLK4 gene comprises the nucleotide sequence provided by HGNC. In some embodiments, the HGNC identifier for KLK4 is 6365. In some embodiments, the KLK4 gene is located at chromosome position 19q13.41. In some embodiments, a KLK4 gene comprises the nucleotide sequence of Ensembl gene identifier ENSG00000167749. In some embodiments, a KLK4 gene comprises the nucleotide sequence of SEQ ID NO:7 or a variant thereof.

Illustrative nucleotide sequences of are provided in Table A.

TABLE A
Illustrative nucleotide sequences of genes of the disclosure.

| SEQ ID | | |
|---|---|---|
| NO | Gene | Sequence |
| 1 | TMPRSS2- | TAGGCGCGAG CTAAGCAGGA GGCGGAGGCG GAGGCGGAGG GCGAGGGGCG |
| | ERG | GGGAGCGCCG CCTGGAGCGC GGCAGGAAGC CTTATCAGTT GTGAGTGAGG |
| | | ACCAGTCGTT GTTTGAGTGT GCCTACGGAA CGCCACACCT GGCTAAGACA |
| | | GAGATGACCG CGTCCTCCTC CAGCGACTAT GGACAGACTT CCAAGATGAG |
| | | CCCACGCGTC CCTCAGCAGG ATTGGCTGTC TCAACCCCCA GCCAGGGTCA |
| | | CCATCAAAAT GGAATGTAAC CCTAGCCAGG TGAATGGCTC AAG |
| 2 | SCHLAP1 | GCTTTTATGA GCTGTAACAC TCACCGCGAA GGTCCGCAGC TTCACTCCTG |
| | | AAGCCAGCGA GACCACGAGC CTACTGGGAG GAACGAACAA CTCCCGACGC |
| | | GCCGCCTTAA GAGCTGTAAC ACTCACCGCG AAGGTCTGCA GCTTCACTCC |
| | | TGAGCCAGCG AGACCACGAA CCCACCAGAA GGAAAAAACT CCGAACACAT |
| | | CTGAACATCA GAAGCAACAA ACTCCGGACA CGCCGCCTTT AAGAACTGTA |
| | | ACACTCACTG CGAGGGTCCG CGGCTTCATT CTTGAAGTGA GTGAGACCAA |
| | | GAACCCACCA GTTCTGGACA CAATTTCAAG TCCTCAGGTG CCATCAATAT |
| | | TCTGAAAATG GCAGTGATTT TTATTCAACC TGTATAAGGC ACTTTCACCA |
| | | TGTACCTGGA AGCAACATCT ACATCTTTTT CAGTTTCTTC TACGCCAGGT |
| | | GTGTGCTTAG CTCCATGACA AAAGGTGACA GCTTATTCTG CAGCACACAC |
| | | ACATCATCAA AGTGGGAGGT GGTGAGACTG GCACACTGAC AGTCTGTCCT |
| | | AGCAGATTTC AGCTCACACT GCAATCTAGA TGCTGGGGAC ACAAGGTCCA |
| | | CCTTCCAGGA ATATGGCCAT GACACCAGAA ATCACAAACA TGATGAGAAT |
| | | GGAATGACTG GGGAAGAAGT GCCAGATGCT TCACTTGTAA ATGAAGACCC |
| | | AGCCTCTGGG GATGCAGATA CCACCTCCCT GAAGAAGCTG AATATCTGCA |
| | | GATAAGTGGA GTTCACCAAT GATGAGGAGC GGGATGGAGA AAGGAGGTAG |
| | | GGAGAGTCAT CCAAGGAACA TGAGCAACAT GTTAAAAGCC AAGTGGTTTA |
| | | ATTTCTGGAG ATGGTGAACC CAAGAGGCTC TGCTGGGAGA CAACAAAAAT |
| | | AATGAAGAAT TGAACCAGAG TCCGGTGAAT ATCAGCACTG GGACCAGTTA |
| | | GCAGAGGAAA AGGAAAGAAT AAAAGCGAAA AGAATGAAGA GTCATATGAT |
| | | TACCAACTTT TCCTTTTTCA TATAAATTGA GTGTATATGG GTCTGGAACA |
| | | ACCTGAATTT CCATCAAGTC CTGGCTAACC TCATTATGTC CTATGAATAT |
| | | TTTTGACTAA TCCCACTTTA CATTAATCTG TATTGTGAAT GTGGATATTG |
| | | AATTATATTT CTTTGTAATC CCATTATCCA AAATCCAGTT CAGAGACTAT |
| | | TAGTTACCAA TGTTCACTGT GAAGGAAAAA AAAAAAAAAA AAGCTCAGAG |
| | | GATAAACATG TGATATGGTT TGGCTGTGTC CCCACCCAAA TATCATCTTG |
| | | AATTGTAGCT CCCATAATTC CCACGTGTTG TGGGAGGGAC CCGGTGGGAG |
| | | ATAATTGTAT CATGGGGGTG GTTCCCCCAT ACTATTCTCA TAGTAGTGAA |
| | | TAAGTCTCAC AAAATCTGAT GGTTTTATGA GGGAAAACCC CTTTCACCTG |
| | | GTTCTCATTC TCTTCTCTGG TCTGTCGTCA TGTAAGACAT GCCTTTCACC |
| | | TTCTCCACCA TGACTGTGAG GCCTCCCCAG CCACGTGGAA CTGTGAGCCC |
| | | ATTAAACCTC TTTCACTTAT AAAT |
| 3 | OR51E2 | CTTCTGGGAA TCTCCACACC CTGAAGACAC AGTGAGTTAG CACCACCACC |
| | | AGGAATTGGC CTTTCAGCTC TGTGCCTGTC TCCAGTCAGG CTGGAATAAG |
| | | TCTCCTCATA TTTGCAAGCT CGGCCCTCCC CTGGAATCTA AAGCCTCCTC |
| | | AGCCTTCTGA GTCAGCCTGA AAGGAACAGG CCGAACTGCT GTATGGGCTC |
| | | TACTGCCAGT GTGACCTCAC CCTCTCCAGT CACCCCTCCT CAGTTCCAGC |
| | | TATGAGTTCC TGCAACTTCA CACATGCCAC CTTTGTGCTT ATTGGTATCC |
| | | CAGGATTAGA GAAAGCCCAT TTCTGGGTTG GCTTCCCCCT CCTTTCCATG |
| | | TATGTAGTGG CAATGTTTGG AAACTGCATC GTGGTCTTCA TCGTAAGGAC |
| | | GGAACGCAGC CTGCACGCTC CGATGTACCT CTTTCTCTGC ATGCTTGCAG |
| | | CCATTGACCT GGCCTTATCC ACATCCACCA TGCCTAAGAT CCTTGCCCTT |
| | | TTCTGGTTTG ATTCCCGAGA GATTAGCTTT GAGGCCTGTC TTACCCAGAT |
| | | GTTCTTTATT CATGCCCTCT CAGCCATTGA ATCCACCATC CTGCTGGCCA |
| | | TGGCCTTTGA CCGTTATGTG GCCATCTGCC ACCCACTGCG CCATGCTGCA |
| | | GTGCTCAACA ATACAGTAAC AGCCCAGATT GGCATCGTGG CTGTGGTCCG |
| | | CGGATCCCTC TTTTTTTTCC CACTGCCTCT GCTGATCAAG CGGCTGGCCT |
| | | TCTGCCACTC CAATGTCCTC TCGCACTCCT ATTGTGTCCA CCAGGATGTA |
| | | ATGAAGTTGG CCTATGCAGA CACTTTGCCC AATGTGGTAT ATGGTCTTAC |
| | | TGCCATTCTG CTGGTCATGG GCGTGGACGT AATGTTCATC TCCTTGTCCT |
| | | ATTTTCTGAT AATACGAACG GTTCTGCAAC TGCCTTCCAA GTCAGAGCGG |
| | | GCCAAGGCCT TTGGAACCTG TGTGTCACAC ATTGGTGTGG TACTCGCCTT |
| | | CTATGTGCCA CTTATTGGCC TCTCAGTGGT ACACCGCTTT GGAAACAGCC |
| | | TTCATCCCAT TGTGCGTGTT GTCATGGGTG ACATCTACCT GCTGCTGCCT |
| | | CCTGTCATCA ATCCCATCAT CTATGGTGCC AAAACCAAAC AGATCAGAAC |
| | | ACGGGTGCTG GCTATGTTCA AGATCAGCTG TGACAAGGAC TTGCAGGCTG |
| | | TGGGAGGCAA GTGACCCTTA ACACTACACT TCTCCTTATC TTTATTGGCT |
| | | TGATAAACAT AATTATTTCT AACACTAGCT TATTTCCAGT TGCCCATAAG |
| | | CACATCAGTA CTTTTCTCTG GCTGGAATAG TAAACTAAAG TATGGTACAT |
| | | CTACCTAAAG GACTATTATG TGGAATAATA CATACTAATG AAGTATTACA |
| | | TGATTTAAAG ACTACAATAA AACCAAACAT GCTTATAACA TTAAGAAAAA |
| | | CAATAAAGAT ACATGATTGA AACCAAGTTG AAAAATAGCA TATGCCTTGG |
| | | AGGAAATGTG CTCAAATTAC TAATGATTTA GTGTTGTCCC TACTTTCTCT |
| | | CTCTTTTTTC TTTCTTTTTT TTTTATTATG GTTAGCTGTC ACATACAACT |
| | | TTTTTTTTTT TTGAGATGGG GTCTCGCTCT GTCACCAGGC TGGAGTGCAG |
| | | TGGCGCGATC TCGGCTCACT GCAACCTCCA CATCCCATGT TGAAGTAATT |
| | | CTTCTGCCTC AGCCTCCCGA GTAGCTGGGA CTAGAGGAAC GTGCCACCAT |
| | | GACTGGCTAA TTTTCTGTAT TTTTTAGTAG AGACAGAGTT TCACCATGTT |
| | | GGCCAGGATG GTCTCGATCT CCTGACCTTG TGATCCACCC GCCTCAGCCT |
| | | CCCAAAGTGT TGGGATTACA GGTGTGAACC ACTGTGCCCG GCCTGTGTAC |
| | | AACTTTTTAA ATAGGGAATA TGATAGCTTC GCATGGTGGT GTGCACCTAT |
| | | AGCCCCCACT GCCTGGAAAG CTGAGGTGGG AGAATCGCTT GAGTCCAGGA |
| | | GTTTGAGGTT ACAGTGATCC ACGATCGTAC CACTACACTC CAGCCTGGGC |
| | | AACAGAGCAA GACCCTGTCT CAAAGCATAA AATGGAATAA CATATCAAAT |
| | | GAAACAGGGA AAATGAAGCT GACAATTTAT GGAAGCCAGG GCTTGTCACA |
| | | GTCTCTACTG TTATTATGCA TTACCTGGGA ATTTATATAA GCCCTTAATA |
| | | ATAATGCCAA TGAACATCTC ATGTGTGCTC ACAATGTTCT GGCACTATTA |
| | | TAAGTGCTTC ACAGGTTTTA TGTGTTCTTC GTAACTTTAT GGAGTAGGTA |
| | | CCATTTGTGT CTCTTTATTA TAAGTGAGAG AAATGAAGTT TATATTATCA |
| | | AGGGGACTAA AGTCACACGG CTTGTGGGCA CTGTGCCAAG ATTTAAAATT |
| | | AAATTTGATG GTTGAATACA GTTACTTAAT GACCATGTTA TATTGCTTCC |
| | | TGTGTAACAT CTGCCATTTA TTTCCTCAGC TGTACAAATC CTCTGTTTTC |
| | | TCTCTGTTAC ACACTAACAT CAATGGCTTT GTACTTGTGA TGAGAGATAA |
| | | CCTTGCCCTA GTTGTGGGCA ACACATGCAG AATAATCCTG TTTTACAGCT |
| | | GCCTTTCGTG ATCTTATTGC TTGCTTTTTT CCAGATTCAG GGAGAATGTT |
| | | GTTGTCTATT TGTCTCTTAC ATCTCCTTGA TCATGTCTTC ATTTTTTAAT |
| | | GTGCTCTGTA CCTGTCAAAA ATTTTGAATG TACACCACAT GCTATTGTCT |
| | | GAACTTGAGT ATAAGATAAA ATAAAATTTT ATTTTAAATT TT |
| 4 | APOC1 | AGGCGGTCAG GGGAAGGCTC AGGAGGAGGG AGATCAACAT CAACCTGCCC |
| | | CGCCCCCTCC CCAGCCTGAT AAAGGTCCTG CGGGCAGGAC AGGACCTCCC |
| | | AACCAAGCCC TCCAGCAAGG ATTCAGAGTG CCCCTCCGGC CTCGCCATGA |
| | | GGCTCTTCCT GTCGCTCCCG GTCCTGGTGG TGGTTCTGTC GATCGTCTTG |
| | | GAAGGCCCAG CCCCAGCCCA GGGGACCCCA GACGTCTCCA GTGCCTTGGA |
| | | TAAGCTGAAG GAGTTTGGAA ACACACTGGA GGACAAGGCT CGGGAACTCA |
| | | TCAGCCGCAT CAAACAGAGT GAACTTTCTG CCAAGATGCG GGAGTGGTTT |
| | | TCAGAGACAT TTCAGAAAGT GAAGGAGAAA CTCAAGATTG ACTCATGAGG |
| | | ACCTGAAGGG TGACATCCCA GGAGGGGCCT CTGAAATTTC CCACACCCCA |
| | | GCGCCTGTGC TGAGGACTCC CTCCATGTGG CCCCAGGTGC CACCAATAAA |
| | | AATCCTACAG AAAA |
| 5 | PCAT14 | GAGATACGGC CTCGTGGGAA GGGAAAGACC TGACCGTCCC CCAGCCCGAC |
| | | ACCCGTAAAG GGTCTGTGCT GAGGAGGATT AGTAAAAGGG GAAGGCCTCT |
| | | TGCAGTTGAG ATAAGAGGAA GGCCTCCGTC TCCTGCATGT CCTTGGGAAT |
| | | GGAATGTCTT GGTGTAAAAC CCGATAGTAC ATTCCTTCTA TTCTGAGAGA |
| | | AGAAAACCAC CCTGTGGCTG GAGGGTGAAG GTACTCTACA GTGTGGTCAT |
| | | TGAGGACAAG TTGACGAGAG AGTCCCAAGT ACGTCCACGG TCAGCCTTGC |
| | | GACATTTAAA GTTCTACAAT GAACTCACTG GAGATGCAAA GAAAAGTGTG |
| | | GAGATGGAGA CACCCCAATC GACTCGCCAG TCTACAGGTG TATCCAGCAG |
| | | CTCCAAAGAG ACAGCAACCA GCAAGAATGG GCCATAGTGA CGATGGTGGT |
| | | TTTGTCAAAA AGAAAAGGGG GGGATATGTA AGGAAAAGAG AGATCAGACT |
| | | TTCACTGTGT CTATGTAGAA AAGGAAGACA TAAGAAACTC CATTTTGATC |
| | | TGTACTAAGA AAAATTGTTT TGCCTTGAGA TGCTGTTAAT CTGTAACTTT |
| | | AGCCCCAACC CTGTGCTCAC GGAAACATGT GCTGTAAGGT TTAAGGGATC |
| | | TAGGGCTGTG CAGGATGTAC CTTGTTAACA ATATGTTTGC AGGCAGTATG |
| | | TTTGGTAAAA GTCATCGCCA TTCTCCATTC TCGATTAACC AGGGGCTCAA |
| | | TGCACTGTGG AAAGCCACAG GAACCTCTGC CCAAGAAAGC CTGGCTGTTG |
| | | TGGGAAGTCA GGGACCCCGA ATGGAGGGAC CAGCTGGTGC TGCATCAGGA |
| | | AACATAAATT GTGAAGATTT CTTGGACATT TATCAGTTTC CAAAATTAAT |
| | | ACTTTTATAA TTTCTTACAC CTGTCTTACT TTAATCTCTT AATCCTGTTA |
| | | TCTTTGTAAG CTGAGGATAT ACGTCACCTC AGGACCACTA TTGTACAAAT |
| | | TGATTGTAAA ACATGTTCAC ATGTGTTTGA ACAATATGAA ATCAGTGCAC |
| | | CTTGAAAATG AACAGAATAA CAGTGATTTT AGGGAACAAA GGAAGACAAC |
| | | CATAAGGTCT GACTGCCTGA GGGGTCGGGC AAAAAGCCAT ATTTTTCTTC |
| | | TTGCAGAGAG CCTATAAATG GACGTGCAAG TAGGAGAGAT ATTGCTAAAT |
| | | T |
| 6 | PCA3 | ACAGAAGAAA TAGCAAGTGC CGAGAAGCTG GCATCAGAAA AACAGAGGGG |
| | | AGATTTGTGT GGCTGCAGCC GAGGGAGACC AGGAAGATCT GCATGGTGGG |
| | | AAGGACCTGA TGATACAGAG GTGAGAAATA AGAAAGGCTG CTGACTTTAC |
| | | CATCTGAGGC CACACATCTG CTGAAATGGA GATAATTAAC ATCACTAGAA |
| | | ACAGCAAGAT GACAATATAA TGTCTAAGTA GTGACATGTT TTTGCACATT |
| | | TCCAGCCCCT TTAAATATCC ACACACACAG GAAGCACAAA AGGAAGCACA |
| | | GAGATCCCTG GGAGAAATGC CCGGCCGCCA TCTTGGGTCA TCGATGAGCC |
| | | TCGCCCTGTG CCTGGTCCCG CTTGTGAGGG AAGGACATTA GAAAATGAAT |
| | | TGATGTGTTC CTTAAAGGAT GGGCAGGAAA ACAGATCCTG TTGTGGATAT |
| | | TTATTTGAAC GGGATTACAG ATTTGAAATG AAGTCACAAA GTGAGCATTA |
| | | CCAATGAGAG GAAAACAGAC GAGAAAATCT TGATGGCTTC ACAAGACATG |
| | | CAACAAACAA AATGGAATAC TGTGATGACA TGAGGCAGCC AAGCTGGGGA |
| | | GGAGATAACC ACGGGGCAGA GGGTCAGGAT TCTGGCCCTG CTGCCTAAAC |
| | | TGTGCGTTCA TAACCAAATC ATTTCATATT TCTAACCCTC AAAACAAAGC |
| | | TGTTGTAATA TCTGATCTCT ACGGTTCCTT CTGGGCCCAA CATTCTCCAT |
| | | ATATCCAGCC ACACTCATTT TTAATATTTA GTTCCCAGAT CTGTACTGTG |
| | | ACCTTTCTAC ACTGTAGAAT AACATTACTC ATTTTGTTCA AAGACCCTTC |
| | | GTGTTGCTGC CTAATATGTA GCTGACTGTT TTTCCTAAGG AGTGTTCTGG |
| | | CCCAGGGGAT CTGTGAACAG GCTGGGAAGC ATCTCAAGAT CTTTCCAGGG |
| | | TTATACTTAC TAGCACACAG CATGATCATT ACGGAGTGAA TTATCTAATC |
| | | AACATCATCC TCAGTGTCTT TGCCCATACT GAAATTCATT TCCCACTTTT |
| | | GTGCCCATTC TCAAGACCTC AAAATGTCAT TCCATTAATA TCACAGGATT |
| | | AACTTTTTTT TTTAACCTGG AAGAATTCAA TGTTACATGC AGCTATGGGA |
| | | ATTTAATTAC ATATTTTGTT TTCCAGTGCA AAGATGACTA AGTCCTTTAT |
| | | CCCTCCCCTT TGTTTGATTT TTTTTCCAGT ATAAAGTTAA AATGCTTAGC |
| | | CTTGTACTGA GGCTGTATAC AGCCACAGCC TCTCCCCATC CCTCCAGCCT |
| | | TATCTGTCAT CACCATCAAC CCCTCCCATG CACCTAAACA AAATCTAACT |
| | | TGTAATTCCT TGAACATGTC AGGCATACAT TATTCCTTCT GCCTGAGAAG |
| | | CTCTTCCTTG TCTCTTAAAT CTAGAATGAT GTAAAGTTTT GAATAAGTTG |
| | | ACTATCTTAC TTCATGCAAA GAAGGGACAC ATATGAGATT CATCATCACA |
| | | TGAGACAGCA AATACTAAAA GTGTAATTTG ATTATAAGAG TTTAGATAAA |
| | | TATATGAAAT GCAAGAGCCA CAGAGGGAAT GTTTATGGGG CACGTTTGTA |
| | | AGCCTGGGAT GTGAAGCAAA GGCAGGGAAC CTCATAGTAT CTTATATAAT |
| | | ATACTTCATT TCTCTATCTC TATCACAATA TCCAACAAGC TTTTCACAGA |
| | | ATTCATGCAG TGCAAATCCC CAAAGGTAAC CTTTATCCAT TTCATGGTGA |
| | | GTGCGCTTTA GAATTTTGGC AAATCATACT GGTCACTTAT CTCAACTTTG |
| | | AGATGTGTTT GTCCTTGTAG TTAATTGAAA GAAATAGGGC ACTCTTGTGA |
| | | GCCACTTTAG GGTTCACTCC TGGCAATAAA GAATTTACAA AGAGCTACTC |
| | | AGGACCAGTT GTTAAGAGCT CTGTGTGTGT GTGTGTGTGT GTGAGTGTAC |
| | | ATGCCAAAGT GTGCCTCTCT CTCTTTGACC CATTATTTCA GACTTAAAAA |
| | | CAAGCATGTT TTCAAATGGC ACTATGAGCT GCCAATGATG TATCACCACC |
| | | ATATCTCATT ATTCTCCAGT AAATGTGATA ATAATGTCAT CTGTTAACAT |
| | | AAAAAAAGTT TGACTTCACA AAAGCAGCTG GAAATGGACA ACCACAATAT |
| | | GCATAAATCT AACTCCTACC ATCAGCTACA CACTGCTTGA CATATATTGT |
| | | TAGAAGCACC TCGCATTTGT GGGTTCTCTT AAGCAAAATA CTTGCATTAG |
| | | GTCTCAGCTG GGGCTGTGCA TCAGGCGGTT TGAGAAATAT TCAATTCTCA |
| | | GCAGAAGCCA GAATTTGAAT TCCCTCATCT TTTAGGAATC ATTTACCAGG |
| | | TTTGGAGAGG ATTCAGACAG CTCAGGTGCT TTCACTAATG TCTCTGAACT |
| | | TCTGTCCCTC TTTGTGTTCA TGGATAGTCC AATAAATAAT GTTATCTTTG |
| | | AACTGATGCT CATAGGAGAG AATATAAGAA CTCTGAGTGA TATCAACATT |
| | | AGGGATTCAA AGAAATATTA GATTTAAGCT CACACTGGTC AAAAGGAACC |
| | | AAGATACAAA GAACTCTGAG CTGTCATCGT CCCCATCTCT GTGAGCCACA |
| | | ACCAACAGCA GGACCCAACG CATGTCTGAG ATCCTTAAAT CAAGGAAACC |
| | | AGTGTCATGA GTTGAATTCT CCTATTATGG ATGCTAGCTT CTGGCCATCT |
| | | CTGGCTCTCC TCTTGACACA TATTAGCTTC TAGCCTTTGC TTCCACGACT |
| | | TTTATCTTTT CTCCAACACA TCGCTTACCA ATCCTCTCTC TGCTCTGTTG |
| | | CTTTGGACTT CCCCACAAGA ATTTCAACGA CTCTCAAGTC TTTTCTTCCA |
| | | TCCCCACCAC TAACCTGAAT GCCTAGACCC TTATTTTTAT TAATTTCCAA |
| | | TAGATGCTGC CTATGGGCTA TATTGCTTTA GATGAACATT AGATATTTAA |
| | | AGCTCAAGAG GTTCAAAATC CAACTCATTA TCTTCTCTTT CTTTCACCTC |
| | | CCTGCTCCTC TCCCTATATT ACTGATTGCA CTGAACAGCA TGGTCCCCAA |
| | | TGTAGCCATG CAAATGAGAA ACCCAGTGGC TCCTTGTGGT ACATGCATGC |
| | | AAGACTGCTG AAGCCAGAAG GATGACTGAT TACGCCTCAT GGGTGGAGGG |
| | | GACCACTCCT GGGCCTTCGT GATTGTCAGG AGCAAGACCT GAGATGCTCC |
| | | CTGCCTTCAG TGTCCTCTGC ATCTCCCCTT TCTAATGAAG ATCCATAGAA |
| | | TTTGCTACAT TTGAGAATTC CAATTAGGAA CTCACATGTT TTATCTGCCC |
| | | TATCAATTTT TTAAACTTGC TGAAAATTAA GTTTTTTCAA AATCTGTCCT |
| | | TGTAAATTAC TTTTTCTTAC AGTGTCTTGG CATACTATAT CAACTTTGAT |
| | | TCTTTGTTAC AACTTTTCTT ACTCTTTTAT CACCAAAGTG GCTTTTATTC |
| | | TCTTTATTAT TATTATTTTC TTTTACTACT ATATTACGTT GTTATTATTT |
| | | TGTTCTCTAT AGTATCAATT TATTTGATTT AGTTTCAATT TATTTTTATT |
| | | GCTGACTTTT AAAATAAGTG ATTCGGGGGG TGGGAGAACA GGGGAGGGAG |
| | | AGCATTAGGA CAAATACCTA ATGCATGTGG GACTTAAAAC CTAGATGATG |
| | | GGTTGATAGG TGCAGCAAAC CACTATGGCA CACGTATACC TGTGTAACAA |
| | | ACCTACACAT TCTGCACATG TATCCCAGAA CGTAAAGTAA AATTTAAAAA |
| | | AAAGTGA |
| 7 | KLK4 | AGGCAGCAGG CTGGAGCTCA GCCCAGCAGT GGAATCCAGG AGCCCAGAGG |
| | | TGGCCGGGTG CTGACGTGAT GGCCACAGCA GGAAATCCCT GGGGCTGGTT |
| | | CCTGGGGTAC CTCATCCTTG GTGTCGCAGG ATCGCTCGTC TCTGGTAGCT |
| | | GCAGCCAAAT CATAAACGGC GAGGACTGCA GCCCGCACTC GCAGCCCTGG |
| | | CAGGCGGCAC TGGTCATGGA AAACGAATTG TTCTGCTCGG GCGTCCTGGT |
| | | GCATCCGCAG TGGGTGCTGT CAGCCGCACA CTGTTTCCAG AACTCCTACA |
| | | CCATCGGGCT GGGCCTGCAC AGTCTTGAGG CCGACCAAGA GCCAGGGAGC |
| | | CAGATGGTGG AGGCCAGCCT CTCCGTACGG CACCCAGAGT ACAACAGACC |
| | | CTTGCTCGCT AACGACCTCA TGCTCATCAA GTTGGACGAA TCCGTGTCCG |
| | | AGTCTGACAC CATCCGGAGC ATCAGCATTG CTTCGCAGTG CCCTACCGCG |
| | | GGGAACTCTT GCCTCGTTTC TGGCTGGGGT CTGCTGGCGA ACGGCAGAAT |
| | | GCCTACCGTG CTGCAGTGCG TGAACGTGTC GGTGGTGTCT GAGGAGGTCT |
| | | GCAGTAAGCT CTATGACCCG CTGTACCACC CCAGCATGTT CTGCGCCGGC |
| | | GGAGGGCAAG ACCAGAAGGA CTCCTGCAAC GGTGACTCTG GGGGGCCCCT |
| | | GATCTGCAAC GGGTACTTGC AGGGCCTTGT GTCTTTCGGA AAAGCCCCGT |
| | | GTGGCCAAGT TGGCGTGCCA GGTGTCTACA CCAACCTCTG CAAATTCACT |
| | | GAGTGGATAG AGAAAACCGT CCAGGCCAGT TAACTCTGGG GACTGGGAAC |
| | | CCATGAAATT GACCCCCAAA TACATCCTGC GGAAGGAATT CAGGAATATC |
| | | TGTTCCCAGC CCCTCCTCCC TCAGGCCCAG GAGTCCAGGC CCCCAGCCCC |
| | | TCCTCCCTCA AACCAAGGGT ACAGATCCCC AGCCCCTCCT CCCTCAGACC |
| | | CAGGAGTCCA GACCCCCCAG CCCCTCCTCC CTCAGACCCA GGAGTCCAGC |
| | | CCCTCCTCCC TCAGACCCAG GAGTCCAGAC CCCCCAGCCC CTCCTCCCTC |
| | | AGACCCAGGA GTCCAGCCCC TCCTCCCTCA GACCCAGGAG TCCAGACCCC |
| | | CCAGCCCCTC CTCCCTCAGA CCCAGGGGTC CAGGCCCCCA ACCCCTCCTC |
| | | CCTCAGACTC AGAGGTCCAG GCCCCCAACC CCTCCTTCCC CAGACCCAGA |
| | | GGTCCAGGTC CCAGCCCCTC CTCCCTCAGA CCCAGCGGTC CAATGCCACC |
| | | TAGACTCTCC CTGTACACAG TGCCCCCTTG TGGCACGTTG ACCCAACCTT |
| | | ACCAGTTGGT TTTTCATTTT TTGTCCCTTT CCCCTAGATC CAGAAATAAA |
| | | GTCTAAGAGA AGCGCA |

In some embodiments, the methods and kits described herein are useful for detecting a level or an amount of expression of TMPRSS2-ERG, SCHLAP1, OR51E2, PCAT14, PCA3, and KLK4 and optionally APOC1.

In some embodiments, the level or amount of expression of TMPRSS2-ERG, SCHLAP1, OR51E2, PCAT14, PCA3, and KLK4 and optionally APOC1, is higher in a subject at risk for having Grade Group ≥2 prostate cancer than in a subject at risk for having or developing a Grade Group <2 prostate cancer or in a subject having no prostate cancer.

### Methods for Detecting Expression of Genes

The level or amount of expression of genes of the present disclosure can be detected using any of a variety of nucleic acid techniques, including but not limited to: nucleic acid sequencing; nucleic acid hybridization; and nucleic acid amplification.

In some embodiments, nucleic acid sequencing methods are utilized (e.g., for detection of amplified nucleic acids). In some embodiments, the technology provided herein finds use in a Second Generation (i.e., Next Generation or Next-Gen), Third Generation (i.e., Next-Next-Gen), or Fourth Generation (i.e., N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92:255 (2008), herein incorporated by reference in its entirety. Those of skill in the art will recognize that because RNA is less stable in the cell and more prone to nuclease attack experimentally RNA can be reverse transcribed to DNA before sequencing.

Suitable nucleic acid sequencing techniques include, but are not limited to, sequencing by synthesis (see e.g., Meyer and Kircher, “Illumina sequencing library preparation for highly multiplexed target capture and sequencing,” Cold Spring Harbor Protocols 2010 (6)); single-molecule real-time sequencing (see e.g., Levene et al., “Zero-Mode Waveguides for Single-Molecule Analysis at High Concentrations,” Science. 299 (5607): 682-6 (2003)); ion semiconductor sequencing (see e.g., Rusk, “Torrents of sequence,” Nat. Methods 8, 44 (2011)); pyrosequencing (see e.g., Wicker et al., “454 sequencing put to the test using the complex genome of barley,” BMC Genomics, 7:275, 2006); sequencing by ligation (SOLID sequencing) (see e.g., Margulies et al., “Genome sequencing in microfabricated high-density picolitre reactors,” Nature, 437:376-80 (2005)); nanopore sequencing (see e.g., Goodwin et al., “Oxford Nanopore sequencing, hybrid error correction, and de novo assembly of a eukaryotic genome,” Genome Res., 25 (11): 1750-6 (2015)); chain termination sequencing (Sanger sequencing) (see e.g., Sanger et al., “DNA sequencing with chain-terminating inhibitors, “Proceedings of the National Academy of Sciences of the United States of America, 74 (12): 5463-5467 (1977)); and sequencing with mass spectrometry (see e.g., Edwards et al., “Mass-spectrometry DNA sequencing,” Mutation Research, 573 (1-2): 3-12 (2005)).

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH can be used to measure and localize mRNAs and other transcripts (e.g., cancer markers) within tissue sections or whole mounts. Sample cells and tissues can be treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

The cancer markers in the methods described herein can be detected by conducting one or more hybridization reactions. The one or more hybridization reactions may comprise one or more hybridization arrays, hybridization reactions, hybridization chain reactions, isothermal hybridization reactions, nucleic acid hybridization reactions, or a combination thereof. The one or more hybridization arrays may comprise hybridization array genotyping, hybridization array proportional sensing, DNA hybridization arrays, macroarrays, microarrays, high-density oligonucleotide arrays, genomic hybridization arrays, comparative hybridization arrays, or a combination thereof.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., cancer markers) by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Nucleic acid variations may be detected by amplification reactions coupled with probe-based detection or sequencing. Nucleic acids may be amplified prior to or simultaneous with detection. Illustrative non-limiting examples of nucleic acid amplification techniques include, but are not limited to, polymerase chain reaction (PCR), TAQMAN amplification, reverse transcription polymerase chain reaction (RT-PCR), quantitative allele-specific real-time target and signal amplification (QuARTS), target enrichment long-probe quantitative amplified signal (TELQAS), transcription-mediated amplification (TMA), ligase chain reaction (LCR), strand displacement amplification (SDA), is recombinase polymerase amplification (RPA) (TwistDx, Maidenhead, UK), transcription mediated amplification, emulsion PCR, bridge amplification, isothermal amplification, single strand displacement amplification, rolling circle amplification, whole genome amplification, helicase dependent amplification, nucleic acid sequence-based amplification (NASBA) or any suitable nucleic acid amplification technology. Those of ordinary skill in the art will recognize that certain amplification techniques (e.g., PCR) typically involve RNA reverse transcription to DNA prior to amplification (e.g., RT-PCR), whereas other amplification techniques directly amplify RNA (e.g., TMA and NASBA).

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89:392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence-based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6:1197 (1988), herein incorporated by reference in its entirety), commonly referred to as QB replicase; a transcription-based amplification method (Kwoh et al., Proc. Natl. Acad. Sci. USA 86:1173 (1989)); and, self-sustained sequence replication (Guatelli et al., Proc. Natl. Acad. Sci. USA 87:1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

In some embodiments, amplification methods are real time quantitative PCR methods (QPCR). A real-time polymerase chain reaction (real-time PCR, or qPCR) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively (quantitative real-time PCR) and semi-quantitatively (i.e., above/below a certain amount of DNA molecules) (semi-quantitative real-time PCR). Two common methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

In some embodiments, the level or amount of mRNA is detected using RT-qPCR analysis which provides Ct (cycle threshold values) for each mRNA detected. In a real-time PCR assay a positive reaction is detected by accumulation of a fluorescent signal. The Ct value is defined as the number of cycles required for the fluorescent signal to cross the threshold (i.e., exceeds the background level). Ct levels are inversely proportional to the amount of target nucleic acid in the sample (i.e., the lower the Ct value the greater the amount of mRNA in the sample).

In some embodiments, the level or amount of expression of any one of the genes described herein is normalized to a level or an amount of expression of a reference gene. In some embodiments, the amount of expression of mRNA is normalized to the level or amount of expression of mRNA of a reference gene. Reference genes suitable for normalization are known to those of skill in the art and include, but are not limited to, KLK3, CYPB561A3, EEF1A2, GAPDH, HPN, KLK2, KLK4, LBH, NUDT8, SPDEF, or TRGV. In some embodiments, the reference gene is KLK3.

Compositions for use in the methods described herein, such as reagent compositions, include, but are not limited to, antibodies, probes, amplification oligonucleotides, and the like.

The compositions and kits can comprise 1 or more, 2 or more, 3 or more, or 4 or more antibodies, probes, pairs of probes, pairs of amplification oligonucleotide, or sequencing primers.

The probes or primers can hybridize to 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 21 or more target molecules. The target molecules may be RNA, DNA, cDNA, mRNA, a portion or fragment thereof or a combination thereof. In some instances, at least a portion of the target molecules are cancer markers. The probes may hybridize to 1 or more, or 2 or more cancer markers disclosed herein.

Typically, the probes or primers comprise a target specific sequence. The target specific sequence may be complementary to at least a portion of the target molecule. The target specific sequence may be at least about 50% or more, 55% or more, 60% or more, 65% or more, 70% or more, 75% or more, 80% or more, 85% or more, 90% or more, 95% or more, 97% or more, 98% or more, or 100% complementary to at least a portion of the target molecule.

The target specific sequence can be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more nucleotides in length. In some instances, the target specific sequence is between about 8 to about 20 nucleotides, 10 to about 18 nucleotides, or 12 to about 16 nucleotides in length.

The compositions and kits can comprise a plurality of probes or primers, wherein the two or more probes of the plurality of probes comprise identical target specific sequences. The compositions and kits may comprise a plurality of probes, wherein the two or more probes of the plurality of probes comprise different target specific sequences.

The probes can further comprise a unique sequence. The unique sequence is noncomplementary to the cancer marker. The unique sequence may comprise a label, barcode, or unique identifier. The unique sequence may comprise a random sequence, nonrandom sequence, or a combination thereof. The unique sequence may be at least about 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 22 or more, 24 or more, 26 or more, 28 or more, 30 or more nucleotides in length. In some instances, the unique sequence is between about 8 to about 20 nucleotides, 10 to about 18 nucleotides, or 12 to about 16 nucleotides in length.

The probes can further comprise a universal sequence. The universal sequence may comprise a primer binding site. The universal sequence may enable detection of the target sequence. The universal sequence may enable amplification of the target sequence. The universal sequence may enable transcription or reverse transcription of the target sequence. The universal sequence may enable sequencing of the target sequence.

The probe or primer compositions of the present disclosure can be provided on a solid support. The solid support can comprise one or more beads, plates, solid surfaces, wells, chips, or a combination thereof. The beads can be magnetic, antibody coated, protein A crosslinked, protein G crosslinked, streptavidin coated, oligonucleotide conjugated, silica coated, or a combination thereof. Examples of beads include, but are not limited to, Ampure beads, AMPure XP beads, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, oligo-dT conjugated beads, silica beads, silica-like beads, anti-biotin microbead, anti-fluorochrome microbead, and BcMag™ Carboxy-Terminated Magnetic Beads.

The compositions and kits can comprise primers and primer pairs capable of amplifying target molecules, or fragments or subsequences or complements thereof. The nucleotide sequences of the target molecules may be provided in computer-readable media (e.g., memory 712 of FIG. 7) for in silico applications and as a basis for the design of appropriate primers for amplification of one or more target molecules.

Primers based on the nucleotide sequences of target molecules can be designed for use in amplification of the target molecules. For use in amplification reactions such as PCR, a pair of primers can be used. The exact composition of the primer sequences is not critical to the disclosure, but for most applications the primers may hybridize to specific sequences of the target molecules or the universal sequence of the probe under stringent conditions, particularly under conditions of high stringency, as known in the art. The pairs of primers are usually chosen so as to generate an amplification product of at least about 15 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 450 or more, 500 or more, 600 or more, 700 or more, 800 or more, 900 or more, or 1000 or more nucleotides. Algorithms for the selection of primer sequences are generally known, and are available in commercial software packages. These primers may be used in standard quantitative or qualitative PCR-based assays to assess transcript expression levels of target molecules. Alternatively, these primers may be used in combination with probes, such as molecular beacons in amplifications using real-time PCR.

The nucleotide sequence of the entire length of the primer does not need to be derived from the target sequence. Thus, for example, the primer may comprise nucleotide sequences at 5′ and/or 3′ termini that are not derived from the target molecule. Nucleotide sequences which are not derived from the nucleotide sequence of the target molecule may provide additional functionality to the primer. For example, they may provide a restriction enzyme recognition sequence or a “tag” that facilitates detection, isolation, purification or immobilization onto a solid support. Alternatively, the additional nucleotides may provide a self-complementary sequence that allows the primer to adopt a hairpin configuration. Such configurations may be necessary for certain primers, for example, molecular beacon and Scorpion primers, which can be used in solution hybridization techniques.

The probes or primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target molecule is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer to allow detection and/or quantitation of a target polynucleotide representing the target molecule of interest. The target polynucleotide may be the expressed target molecule RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different target molecules may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g., biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the target molecule. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a target polynucleotide may comprise a biotin-binding species, and an optically detectable label may be conjugated to biotin and then bound to the labeled target polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Coding schemes may optionally be used, comprising encoded particles and/or encoded tags associated with different polynucleotides of the disclosure. A variety of different coding schemes are known in the art, including fluorophores, including SCNCs, deposited metals, and RF tags.

### Subjects and Samples

The methods and kits described herein are suitable for detecting a level or an amount of expression of one or more of the genes described herein in a sample from a subject. In some embodiments, a subject from whom a sample is obtained can be selected by the skilled practitioner. In some embodiments, selection of the subject is based upon consideration or analysis of one or more factors. Such factors for consideration include, but are not limited to, family history of a specific disease, genetic predisposition for the disease, increased risk for the disease, physical symptoms which indicate the disease, or environmental reasons. Environmental reasons can include, but are not limited to, lifestyle or exposure to agents which cause or contribute to the specific disease. In some embodiments, selection of a subject is based on the subject's previous history with the disease, positive diagnosis prior to therapy or after therapy, treatment for the disease, or remission or recovery from the disease.

In some embodiments, samples for use with the kits and in the methods of the present disclosure comprise nucleic acids suitable for providing RNA expression information. In principle, the biological sample from which the expressed RNA is obtained and analyzed for target molecule expression can be any material suspected of comprising cancer tissue or cells. The sample can be a biological sample used directly in a method of the disclosure. Alternatively, the sample can be a sample prepared from a biological sample.

In some embodiments, the sample or portion of the sample comprising or suspected of comprising cancer tissue or cells can be any source of biological material, including cells, tissue, secretions, or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. Alternatively, or additionally, the source of the sample can be urine, bile, excrement, sweat, tears, spinal fluid, and stool. In some embodiments, the sources of the sample are secretions. In some embodiments, the secretions are exosomes. In some embodiments, the sample is a urine sample. In some embodiments, the urine sample is obtained after a subject's digital rectal examination (DRE). In some embodiments, the urine sample is obtained within 30 minutes after a subject's DRE. In some embodiments, the urine sample is obtained from 30 minutes to 60 minutes after a subject's DRE. In some embodiments, the urine sample is obtained from 30 minutes to 180 minutes after a subject's DRE. In some embodiments, the urine sample is obtained within one hour after a subject's DRE. In some embodiments, the urine sample is obtained within two hours after a subject's DRE. In some embodiments, the urine sample is obtained within three hours after a subject's DRE. In some embodiments, a urine sample is obtained from a subject who has not had a DRE.

Without wishing to be bound by theory, it is believed that the DRE increases the sample's, e.g., urine sample's, concentration of the mRNA or protein expressed by TMPRSS2-ERG, SCHLAP1, OR51E2, PCAT14, PCA3, and KLK4 and optionally APOC1. This increased concentration facilitates detection of mRNA or protein expressed by the genes.

In some embodiments, a sample is combined with a buffer, e.g., for processing. In some embodiments, the amount of expression of genes described herein is determined from a composition, e.g., a solution or suspension, comprising the sample and a buffer. Buffers suitable for samples are known to those of skill in the art and can be determined based on the type of sample being collected. In some embodiments, the composition further comprises a preservative for adequate stability of the sample. In some embodiments, the buffer to sample ratio is 2:5. In some embodiments, the buffer to sample ratio is 1:5, 2:5, 3:5 or 4:5.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example, an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, PA). Prior to molecular analysis, the embedding material may be removed via any suitable techniques, as known in the art. For example, where the sample is embedded in wax, the embedding material may be removed by extraction with organic solvent(s), for example, xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

In some embodiments, a subject is prostate biopsy naïve, i.e., the subject has not had a prostate biopsy. In some embodiments, a subject has had a prior negative prostate biopsy result. In some embodiments, the prostate biopsy result is negative for Grade Group ≥2 prostate cancer. In some embodiments, one or more additional clinical variables are associated with the subject. In some embodiments, the methods comprise assaying one or more additional clinical variables (e.g., including but not limited to, the subject's prostate volume, PSA level or amount, PSA density, biopsy Gleason score, race, family history of prostate cancer, previous negative prostate biopsy, or abnormal DRE. In some embodiments, one or more additional clinical variables are associated with a subject that had a prior negative prostate biopsy result.

### Determining Likelihood of Having or Developing Grade Group ≥2 Prostate Cancer

In some embodiments, the level or amount of expression of TMPRSS2-ERG, SCHLAP1, OR51E2, PCAT14, PCA3, and KLK4 and optionally APOC1 determines the likelihood of detecting prostate cancer in a subject. In some embodiments, the likelihood of detecting prostate cancer in a subject is based on a prostate biopsy of the subject. In some embodiments, the level or amount of expression of TMPRSS2-ERG, SCHLAP1, OR51E2, PCAT14, PCA3, and KLK4 and optionally APOC1 determines the likelihood of detecting Grade Group ≥2 prostate cancer in a subject. In some embodiments, the likelihood is presented as a score based on the amount or level of expression of TMPRSS2-ERG, SCHLAP1, OR51E2, PCAT14, PCA3, and KLK4 and optionally APOC1 present in a sample from a subject. In some embodiments, the likelihood of detecting Grade Group ≥2 prostate cancer is provided as a score ranging from 0% to 100%. In some embodiments, the likelihood of detecting Grade Group ≥2 prostate cancer is provided as a score ranging from 0.0 to 100.0. In some embodiments, a biopsy naïve subject receiving a score of 0-7.5% means that there is a low risk or low likelihood that Grade Group ≥2 prostate cancer would be detected from a prostate biopsy of the subject. In some embodiments, a subject with a prior negative prostate biopsy result receiving a score of 0-5.4% means that there is a low risk or low likelihood that Grade Group ≥2 prostate cancer would be detected from a prostate biopsy of the subject. In some embodiments, a biopsy naïve subject receiving a score of ≥7.6% means that there is a high risk or high likelihood that Grade Group ≥2 prostate cancer would be detected from a prostate biopsy of the subject. In some embodiments, a subject with a prior negative prostate biopsy result receiving a score of ≥5.5% has a high risk or high likelihood that Grade Group ≥2 prostate cancer would be detected from a prostate biopsy of the subject.

In some embodiments, a computer-based analysis program (e.g., stored in memory 712 and executed by processor 711 of FIG. 7) is used to translate the raw data generated by a detection assay (e.g., the presence, absence, or amount of TMPRSS2-ERG, SCHLAP1, OR51E2, PCAT14, PCA3, and KLK4 and optionally APOC1) into data of predictive value for a clinician, subject or subject's healthcare provider. The clinician, subject or subject's healthcare provider can access the raw data using any suitable means. Thus, in some embodiments, the present disclosure provides the further benefit that the clinician, subject or subject's healthcare provider, who might not be trained in genetics or molecular biology, need not understand the raw data. The data can be presented directly to the clinician subject or subject's healthcare provider in its most useful form. This enables the clinician or healthcare provider to immediately utilize the information in order to optimize the care of the subject.

The information can be received, processed or transmitted to or from one or more laboratories conducting the assays, information providers, medical personnel, or subjects using any suitable method. For example, in some embodiments of the present disclosure, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject can visit a medical center to have the sample obtained and sent to the profiling center, or the subject itself can collect the sample (e.g., a urine sample) and directly send it to a profiling center. Where the sample comprises previously determined biological information, the information can be directly sent to the profiling service by the subject (e.g., an information card containing the information may be scanned by a computer (e.g., compute device 701 of FIG. 7) and the data transmitted to a computer (e.g., compute device 701 of FIG. 7) of the profiling center using an electronic communication systems). Once received by the profiling service, the sample can be processed and a profile can be produced (i.e., expression data), useful for the diagnostic or prognostic information desired for the subject.

The profile data is then prepared in a format suitable for interpretation by one or more medical personnel (e.g., a treating clinician, physician assistant, nurse, or pharmacist). For example, rather than providing raw expression data, the prepared format may represent a diagnosis or risk assessment (e.g., levels of the cancer markers described herein) for the subject, along with recommendations for particular treatment options. The data may be displayed to the medical personnel by any suitable method. For example, in some embodiments, the profiling service generates a report that can be printed for the medical personnel (e.g., at the point of care) or displayed to the medical personnel on a computer monitor.

In some embodiments, the information is first analyzed at the point of care or at a regional facility. The raw data is then sent to a central processing facility for further analysis and/or to convert the raw data to information useful for medical personnel or subject. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the medical personnel, the subject, or researchers.

In some embodiments, the subject or the subject's healthcare provider is able to directly access the data using the electronic communication system. The subject may choose further intervention or counseling based on the results.

In some embodiments, the data is used for research use. For example, the data may be used to further optimize the inclusion or elimination of markers as useful indicators of a particular condition or stage of disease or as a companion diagnostic to determine a treatment course of action.

In some embodiments, the level or amount of expression of TMPRSS2-ERG, SCHLAP1, OR51E2, PCAT14, PCA3, and KLK4 and optionally APOC1 is used for determining a score. In some embodiments, determining the score comprises performing an algorithm that generates the score. In some embodiments, the score correlates with or informs the subject's likelihood of having or developing Grade Group ≥2 prostate cancer. In some embodiments, the score indicates a likelihood that Grade Group ≥2 prostate cancer would be detected from a prostate biopsy of the subject. An algorithm to determine the score with an acceptable diagnostic accuracy may be derived based on, for example and without limitation, logistic regression with stepwise feature selection, logistic regression with recursive feature elimination, and regularized logistic regression with elastic net. In some embodiments, performing the algorithm comprises using a processor.

The methods disclosed herein can also comprise transmitting the data/information. For example, data/information derived from the detection and/or quantification of the target may be transmitted to another device and/or instrument. In some instances, the information obtained from an algorithm may also be transmitted to another device and/or instrument. Transmission of the data/information may comprise the transfer of data/information from a first source to a second source. The first and second sources may be in the same approximate location (e.g., within the same room, building, block, campus). Alternatively, first and second sources may be in multiple locations (e.g., multiple cities, states, countries, continents, etc.).

Transmission of the data/information can comprise digital transmission or analog transmission. Digital transmission may comprise the physical transfer of data (a digital bit stream) over a point-to-point or point-to-multipoint communication channel. Examples of such channels are copper wires, optical fibers, wireless communication channels, and storage media. The data may be represented as an electromagnetic signal, such as an electrical voltage, radiowave, microwave, or infrared signal.

Analog transmission may comprise the transfer of a continuously varying analog signal. The messages can either be represented by a sequence of pulses by means of a line code (baseband transmission), or by a limited set of continuously varying wave forms (passband transmission), using a digital modulation method. The passband modulation and corresponding demodulation (also known as detection) can be carried out by modem equipment. According to the most common definition of digital signal, both baseband and passband signals representing bit-streams are considered as digital transmission, while an alternative definition only considers the baseband signal as digital, and passband transmission of digital data as a form of digital-to-analog conversion.

In some embodiments, a report is generated comprising a score. In some embodiments, the score indicates a likelihood that Grade Group ≥2 prostate cancer would be detected from a prostate biopsy of the subject. In some embodiments, the report is accessible by or provided to the subject's healthcare provider. In some embodiments, the report is accessible or provided as a digital or paper copy. In some embodiments, the report is delivered to the subject's healthcare provider by a digital format as described herein (e.g., via electronic mail), or via a courier if the report is in paper copy.

In some embodiments, the report comprises a treatment option. In some embodiments, the report comprises treatment options for Grade Group ≥2 prostate cancer.

### Diagnostic Accuracy

Diagnostic accuracy of the methods or kits described herein can be determined by analyzing the Arca Under the Curve (AUC) derived from Receiver Operator Characteristic (ROC) curves. ROC curves are graphical plots that illustrate the ability of a binary classifier system as its discrimination threshold is varied. ROC curves are plotted with true positive rate against the false positive rate, with true positive rate on the y-axis and false positive rate on the x-axis. The true positive rate, also referred to as the sensitivity, is calculated by dividing the number of true positives by the sum of true positives and false negatives. The false positive rate is calculated by either (1) dividing the number of false positives by the sum of true negatives and false positives, or (2) subtracting the specificity from one, wherein specificity is calculated by dividing the number of true negatives by the sum of true negatives and false positives. In some embodiments, ROC curves are generated based on individual amounts of expression of each gene. In some embodiments, ROC curves are generated based on a combination of amounts of expression of each gene.

In some embodiments, the AUC value of the methods or kits described herein is greater than 0.50. In some embodiments, the AUC value of the methods or kits described herein is at least 0.60. In some embodiments, the AUC value of the methods or kits described herein is at least 0.70. In some embodiments, the AUC value of the methods or kits described herein is at least 0.71. In some embodiments, the AUC value of the methods or kits described herein is at least 0.72. In some embodiments, the AUC value of the methods or kits described herein is at least 0.73. In some embodiments, the AUC value the methods or kits described herein is at least 0.74. In some embodiments, the AUC value of the methods or kits described herein is at least 0.75. In some embodiments, the AUC value of the methods or kits described herein is at least 0.76. In some embodiments, the AUC value of the methods or kits described herein is at least 0.77. In some embodiments, the AUC value of the methods or kits described herein is at least 0.78. In some embodiments, the AUC value of the methods or kits described herein is at least 0.79. In some embodiments, the AUC value of the methods or kits described herein is at least 0.80. In some embodiments, the AUC value of the methods or kits described herein is at least 0.81. In some embodiments, the AUC value of the methods or kits described herein is at least 0.82. In some embodiments, the AUC value of the methods or kits described herein is at least 0.83. In some embodiments, the AUC value of the methods or kits described herein is at least 0.84. In some embodiments, the AUC value of the methods or kits described herein is at least 0.85. In some embodiments, the AUC value of the methods or kits described herein is at least 0.86. In some embodiments, the AUC value of the methods or kits described herein is at least 0.87. In some embodiments, the AUC value of the methods or kits described herein is at least 0.88. In some embodiments, the AUC value of the methods or kits described herein is at least 0.89. In some embodiments, the AUC value of the methods or kits described herein is at least 0.90.

Diagnostic accuracy of the amount of expression of an individual gene or combination of amounts of expression of specific genes can be maximized by implementing a cut-off analysis that takes into account the sensitivity, specificity, negative predictive value (NPV), positive predictive value (PPV), positive likelihood ratio (PLR) and negative likelihood ratio (NLR) necessary for clinical utility. Results of amounts of expression are analyzed in any of a variety of ways. In some embodiments, the results are analyzed using a univariate, or single-variable analysis (SV). In some embodiments, the results are analyzed using multivariate analysis (MV).

The generation of ROC curves and analysis of a population of samples can be used to establish the cutoff value used to distinguish between different subject sub-groups. For example, the cutoff value can be used to distinguish between a high likelihood of detecting Grade Group ≥2 prostate cancer from a subject's prostate biopsy and a low likelihood of detecting Grade Group ≥2 prostate cancer from a subject's prostate biopsy. In some embodiments, the cutoff value can distinguish between these subjects. In some embodiments, the cutoff value may distinguish between subjects with a non-aggressive cancer from an aggressive cancer.

In some embodiments, the methods or kits described herein provide a score indicating with a diagnostic accuracy of at least 0.70 the likelihood that Grade Group ≥2 prostate cancer would be detected from a subject's prostate biopsy. In some embodiments, the methods or kits described herein provide a score indicating with a diagnostic accuracy of at least 0.75 the likelihood that Grade Group ≥2 prostate cancer would be detected from a subject's prostate biopsy. In some embodiments, the methods or kits described herein provide a score indicating with a diagnostic accuracy of at least 0.80 the likelihood that Grade Group ≥2 prostate cancer would be detected from a subject's prostate biopsy.

In some embodiments, the methods or kits described herein provide a score indicating with a diagnostic accuracy of at least 0.70 the likelihood that Grade Group ≥2 prostate cancer would be detected from a prostate biopsy naïve subject's prostate biopsy. In some embodiments, the methods or kits described herein provide a score indicating with a diagnostic accuracy of at least 0.75 the likelihood that Grade Group ≥2 prostate cancer would be detected from a prostate biopsy naïve subject's prostate biopsy. In some embodiments, the methods or kits described herein provide a score indicating with a diagnostic accuracy of at least 0.80 the likelihood that Grade Group 22 prostate cancer would be detected from a prostate biopsy naïve subject's prostate biopsy.

In some embodiments, the methods or kits described herein provide a score indicating with a diagnostic accuracy of at least 0.70 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.75 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.80 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.81 the likelihood that Grade Group ≥2 prostate cancer would be detected from a prostate biopsy of a subject having a previous negative prostate biopsy. In some embodiments, the methods or kits described herein provide a score indicating with a diagnostic accuracy of at least 0.82 the likelihood that Grade Group ≥2 prostate cancer would be detected from a prostate biopsy of a subject having a previous negative prostate biopsy.

In some embodiments, each referenced diagnostic accuracy is achievable where the urine sample is obtained within one hour after a subject's digital rectal examination (DRE). In some embodiments, each referenced diagnostic accuracy is achievable where the urine sample is obtained from 30 minutes to 60 minutes after a subject's DRE. In some embodiments, the urine sample is obtained from 30 minutes to 180 minutes after a subject's DRE. In some embodiments, the urine sample is obtained within one hour after a subject's DRE. In some embodiments, the urine sample is obtained within two hours after a subject's DRE. In some embodiments, the urine sample is obtained within three hours after a subject's DRE. In some embodiments, a urine sample is obtained from a subject who has not had a DRE,

### Kits and Devices

In some embodiments, the disclosure provides kits for analyzing a cancer, comprising (a) a probe set comprising a plurality of probes comprising target specific sequences complementary to TMPRSS2-ERG, SCHLAP1, OR51E2, PCAT14, PCA3, and KLK4 and optionally APOC1, wherein the target molecules comprise TMPRSS2-ERG, SCHLAP1, OR51E2, PCAT14, PCA3, and KLK4 and optionally APOC1; and (b) a computer model or algorithm (e.g., stored in memory 712 and executed by processor 711 of FIG. 7) for analyzing an expression level and/or expression profile of the target molecules in a sample. The target molecules may comprise those described herein or a combination thereof.

In some embodiments, the disclosure provides kits for analyzing a cancer, comprising (a) a probe set comprising a plurality of probes comprising target specific sequences complementary to TMPRSS2-ERG, SCHLAP1, OR51E2, PCAT14, PCA3, and KLK4 and optionally APOC1; and (b) a computer model or algorithm (e.g., stored in memory 712 and executed by processor 711 of FIG. 7) for analyzing an expression level and/or expression profile of the target molecules in a sample. Control samples and/or nucleic acids may optionally be provided in the kit. Control samples may include tissue and/or nucleic acids obtained from or representative of tumor samples from a healthy subject, as well as tissue and/or nucleic acids obtained from or representative of tumor samples from subjects diagnosed with cancer.

Instructions for using the kits to perform one or more methods of the disclosure can be provided, and can be provided in any fixed medium. The instructions may be located inside or outside a container or housing, and/or may be printed on the interior or exterior of any surface thereof. A kit may be in multiplex form for concurrently detecting and/or quantitating target polynucleotides representing the expressed target molecules.

In some embodiments, the disclosure provides kits comprising a container comprising a reagent composition for detecting an amount of expression of TMPRSS2-ERG, SCHLAP1, OR51E2, PCAT14, PCA3, and KLK4 and optionally APOC1; and instructions for detecting the amount of expression. In some embodiments, the reagent composition comprises a polynucleotide reagent for detecting the amount of mRNA expressed by genes. In some embodiments, the reagent composition comprises a polynucleotide reagent for detecting an amount of expression of a reference gene, and the instructions are additionally for normalizing the amount of expression of the genes to the amount of expression of the reference gene. In some embodiments, the instructions are additionally for generating a report comprising a score determined by the amount of expression of the genes, wherein the score indicates the likelihood that Grade Group ≥2 prostate cancer would be detected from a subject's prostate biopsy.

Devices useful for performing methods of the disclosure are also provided. The devices can comprise means for characterizing the expression level of a target molecule of the disclosure, for example components for performing one or more methods of nucleic acid extraction, amplification, and/or detection. Such components may include one or more of an amplification chamber (for example, a thermal cycler), a plate reader, a spectrophotometer, capillary electrophoresis apparatus, a chip reader, and or robotic sample handling components. These components ultimately can obtain data that reflects the expression level of the target molecules used in the assay being employed.

The devices can include an excitation and/or a detection means. Any instrument that provides a wavelength that can excite a species of interest and is shorter than the emission wavelength(s) to be detected can be used for excitation. Commercially available devices can provide suitable excitation wavelengths as well as suitable detection component.

Illustrative excitation sources include a broadband UV light source such as a deuterium lamp with an appropriate filter, the output of a white light source such as a xenon lamp or a deuterium lamp after passing through a monochromator to extract out the desired wavelength(s), a continuous wave (cw) gas laser, a solid-state diode laser, or any of the pulsed lasers. Emitted light can be detected through any suitable device or technique; many suitable approaches are known in the art. For example, a fluorimeter or spectrophotometer may be used to detect whether the test sample emits light of a wavelength characteristic of a label used in an assay.

The devices can comprise a means for identifying a given sample, and of linking the results obtained to that sample. Such means can include manual labels, barcodes, and other indicators which can be linked to a sample vessel, and/or may optionally be included in the sample itself, for example where an encoded particle is added to the sample. The results may be linked to the sample, for example in a computer memory (e.g., memory 712 of FIG. 7) that contains a sample designation and a record of expression levels obtained from the sample. Linkage of the results to the sample can also include a linkage to a particular sample receptacle in the device, which is also linked to the sample identity.

The devices can also comprise a means for correlating the expression levels of the target molecules being studied with a prognosis of disease outcome. Such means may comprise one or more of a variety of correlative techniques, including lookup tables, algorithms, multivariate models, and linear or nonlinear combinations of expression models or algorithms. The expression levels may be converted to one or more likelihood scores, reflecting a likelihood that the subject providing the sample may exhibit a particular disease outcome. The models and/or algorithms can be provided in machine readable format and can optionally further designate a treatment modality for a subject or class of subjects.

The devices can also comprise output means for outputting the disease status, prognosis and/or a treatment modality. Such output means can take any form which transmits the results to a subject and/or a healthcare provider, and may include a monitor, a printed format, or both. The device may use a computer system for performing one or more of the steps provided, such as system 700 of FIG. 7.

### II. Prognosis, Diagnosis or Treatment

The methods, compositions, and kits disclosed herein are useful for the prognosis, diagnosis, predication, monitoring and/or treatment of cancer (e.g., prostate cancer, and in some embodiments Grade Group ≥2 prostate cancer) in a subject. In some embodiments, the predicting, and/or monitoring the status or outcome of a cancer includes assessing the presence or risk of high-grade prostate cancer (i.e., Grade Group ≥2 prostate cancer). In some embodiments, predicting, and/or monitoring the status or outcome of a cancer comprises determining the efficacy of treatment. In some embodiments, methods and kits disclosed herein are useful for indicating the likelihood that Grade Group ≥2 prostate cancer would be detected from a subject's prostate biopsy.

In some embodiments, the methods comprise determining, recommending or administering a therapeutic regimen. In some embodiments, the therapeutic regimen is an anti-cancer therapy. In some embodiments, the methods comprise modifying a therapeutic regimen. Modifying a therapeutic regimen can comprise increasing a therapeutic dosage, decreasing a therapeutic dosage, or terminating a therapeutic regimen.

For example, in some embodiments, the methods described herein are useful to identify subjects with high-grade prostate cancer. In some embodiments, the methods described herein are useful to identify subjects with a high likelihood of having high-grade prostate cancer detectable from a prostate biopsy. Such subjects can be administered prostate cancer therapy (e.g., one or more of surgery, radiation therapy, hormonal therapy, targeted therapy, chemotherapy, immunotherapy, radiopharmaceuticals, or bone-modifying drugs).

Conversely, in some embodiments, subjects identified as having a low-grade prostate cancer, or having a low likelihood of having high-grade prostate cancer, e.g., based on the levels of expression of the described markers, can be given an option to avoid a biopsy or treatment and opt for watchful waiting or minimal treatments.

In some embodiments, the prostate cancer therapy comprises administering a chemotherapeutic agent. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the biological therapy is immune checkpoint therapy. Immune checkpoint inhibitors target CTLA-4, PD-1, or PD-L1. Examples include but are not limited to, ipilimumab, nivolumab, cemiplimab, avelumab, durvalumab, tremelimumab, dostarlimab, pembrolizumab, spartalizumab, and atezolizumab.

In some embodiments, the prostate cancer therapy is FDA-approved for treating prostate cancer. In some embodiments, the prostate cancer therapy is: abiraterone acetate, apulutamide, bicalutamide, cabazitaxel, casodex, darolutamide, degarelix, docetaxel, eligard, enzalutamide, erleada, firmagon, flutamide, goserelin acetate, jevtana, leuprolide acetate, Lupron depot, lutetium lu 177 vipivotide tetraxetan, Lynparza, mitoxantrone hydrochloride, nilandron, nilutamide, nubeqa, Olaparib, orgovyx, pluvicto, provenge, radium 223 dichloride, relugolix, rubraca, rucaparib camsylate, sipuleucel-t, taxotere, xofigo, xtandi, yonsa, zoladex, xytiga, or any combination thereof.

FIG. 7 is a schematic block diagram of an example system 700 that includes a compute device 701 that can be used to implement methods described herein, according to an embodiment. The compute device 701 can be a hardware-based computing device, computer and/or a multimedia device, such as, for example, a device, a desktop computer, a smartphone, a tablet, a wearable device, a laptop computer, a server, and/or the like. The compute device 701 includes a processor 711, a memory 712 (e.g., including data storage), and a communicator 713 (e.g., operatively coupled to one another via a system bus). Where a method includes multiple compute devices, each of those multiple compute devices can be similar or identical in structure and/or function to compute device 701.

The memory 712 (also referred to herein as computer-readable media and/or processor-readable media) of the compute device 701 can be, for example, a random-access memory (RAM), a memory buffer, a hard drive, a read-only memory (ROM), an erasable programmable read-only memory (EPROM), and/or the like. The memory 712 can be configured to store, for example, data. In some instances, the memory 712 can store, for example, one or more software programs and/or code that can include instructions to cause the processor 711 to perform one or more processes, functions, and/or the like (e.g., the processes and/or functions described herein). In some embodiments, the memory 712 can include extendable storage units that can be added and used incrementally. In some implementations, the memory 712 can be a portable memory (e.g., a flash drive, a portable hard disk, and/or the like) that can be operatively coupled to the processor 711. In some instances, the memory can be remotely operatively coupled with the compute device. For example, a remote database device can serve as a memory and be operatively coupled to the compute device.

The communicator 713 can be a hardware device operatively coupled to the processor 711 and memory 712 and/or software stored in the memory 712 executed by the processor 711. The communicator 713 can be, for example, a network interface card (NIC), a Wi-Fi™ module, a Bluetooth® module and/or any other suitable wired and/or wireless communication device. Furthermore, the communicator 713 can include a switch, a router, a hub and/or any other network device. The communicator 713 can be configured to connect the compute device 701 to a communication network (not shown in FIG. 7). In some instances, the communicator 713 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the communicator 713 can facilitate receiving and/or transmitting data or files through a communication network. In some instances, received data and/or a received file can be processed by the processor 711 and/or stored in the memory 712.

The processor 711 can be, for example, a hardware based integrated circuit (IC) or any other suitable processing device configured to run and/or execute a set of instructions or code. For example, the processor 711 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 711 can be operatively coupled to the memory 712 through a system bus (e.g., address bus, data bus and/or control bus).

In use, in some implementations the processor 711 of the compute device 701 can receive data used in the processes, methods and/or algorithms described herein (e.g., analysis program, data processing, data transmission, data analysis, data correlation, algorithms, machine learning models, score generation, data output, prediction, etc.). The data can be received from a user of the compute device 701, from the memory 712, via another database and/or device (e.g., via communicator 713 and a network) and/or from any other suitable data source. The processor 711 can execute and/or implement the processes, methods and/or algorithms described herein (e.g., analysis program, data processing, data transmission, data analysis, data correlation, algorithms, machine learning models, score generation, data output, prediction, etc.). In some implementations, the processor 711 can generate a report based on the score and store the report in the memory 712 and/or present the report to a user (e.g., via a display of compute device 701 or by sending the report to another compute device via the communicator 713 and a network).

### EXPERIMENTAL

The following Examples are provided in order to demonstrate and further illustrate certain embodiments and aspects of the present disclosure and are not to be construed as limiting the scope thereof.

### Example 1

### Methods

### Development Cohort

The same development cohort used to build the original MPS2 models was used [5]. Briefly, prebiopsy urine samples (first-catch urine following digital rectal examination) were prospectively collected at the University of Michigan from patients presenting for 12-core or greater prostate biopsy due to elevated PSA levels (3-10 ng/mL) from 2008 to 2020. A total of 761 samples were included in the final development cohort.

### External Validation Cohort

The external validation cohort was the same one used in the original MPS2 study [5]. The cohort consisted of 743 patients in the prospective NCI EDRN PCA3 Evaluation Trial [7]. This trial enrolled consecutive patients presenting for biopsy across 11 academic centers, primarily due to elevated PSA levels or abnormal digital rectal examination findings.

### Data Preprocessing of Gene Expression Data

Using qPCR profiling from OpenArray™, gene expression was measured in each urine sample via the cycle threshold (Ct), or the number of amplification cycles required for sample fluorescence to exceed the background level. Lower Ct values suggest higher gene expression. In this work, analysis was focused on the 54 genes nominated in the MPS2 study [5]. This expression data was preprocessed by following a similar data preprocessing procedure as taken in the original development of MPS2 [5]. In this procedure, the upper Ct value limit is set to 35. Specifically, Ct values greater than this limit were considered undetected and set to 35. Ct values from OpenArray™ that were “Undetermined” or “Inconclusive/No Amp” were also considered to be undetected and set to the upper Ct value limit of 35. Next, the standard deviation (SD) was computed across 3 technical replicates. If SD>=1, the replicate farthest from the mean was removed; otherwise, all 3 replicates were kept. The average Ct value across these kept technical replicates was then calculated. As a filter for poor quality samples, all samples with an average Ct value of the reference gene KLK3 above the 95thpercentile were excluded. The average Ct values were next normalized for each target gene by KLK3 using the formula-[average Ct of gene X-average Ct of KLK3]. Finally, z-score scaling was applied to the normalized average Ct before downstream model development and feature selection. This data preprocessing pipeline is referred to as the base preprocessing pipeline.

While the base preprocessing pipeline serves as a reasonable starting point, it is important to note that human judgment calls or choices such as the procedure for handling undetectable Ct values and poor quality samples were inevitably made. Hence, to improve the robustness of the model and conclusions against these preprocessing choices, three alternative data preprocessing pipelines were examined, each equally-reasonable but a slight modification of the base preprocessing pipeline.
1. Ct limit=40 preprocessing pipeline: Rather than setting the upper Ct value limit to 35 for undetected replicates, the upper Ct value limit was set to 40. All other preprocessing steps remain unchanged from the base preprocessing pipeline.
2. Normalized Ct limit=−21 preprocessing pipeline: In the aforementioned data preprocessing pipelines, the Ct values for undetected replicates were set prior to the normalization of the Ct values. Thus, the normalized Ct value for undetected replicates differs between genes. For comparison, in this preprocessing pipeline, the Ct values for all undetected replicates after Ct normalization were replaced to have a constant value of −21 (which was the lowest Ct value post-normalization). All other preprocessing steps remain unchanged from the base preprocessing pipeline.
3. No sample exclusion preprocessing pipeline: Rather than excluding all samples with an average Ct value of the reference gene KLK3 above the 95thpercentile, this preprocessing pipeline does not exclude any samples based upon their Ct value for the reference gene KLK3. All other data preprocessing steps remain unchanged from the base preprocessing pipeline.

### Modeling Choices

Many different statistical and/or machine learning models were considered for predicting high-grade PCa from the preprocessed gene expression data. Namely, common statistical or machine learning models such as ordinary logistic regression, logistic regression with L1 (LASSO) regularization [8], L2 (ridge) regularization [9], and combined L1+L2 (clastic net) regularization [10], random forests (RF) [11], gradient boosting decision trees [12], and RuleFit were considered. Recently developed tree-based machine learning methods including random forest+ (RF+) [14], a PCS-guided generalization of random forests which combines the strength of both linear models and nonlinear trees, and fast interpretable greedy-tree sums (FIGS.) [15], which grows a flexible but controllable number of shallow decision trees in summation were also considered. These interpretable linear and tree-based models were used for identifying important genes for reliable biomarker development. Tree-based machine learning models are often well-suited for biological tasks such as this, in part due to the resemblance between the thresholding behavior of decision trees and the on-off switch-like behavior commonly thought to govern genetic processes [16]. Specific implementations of each model and their hyperparameters are detailed in Table 1.

### Model Development

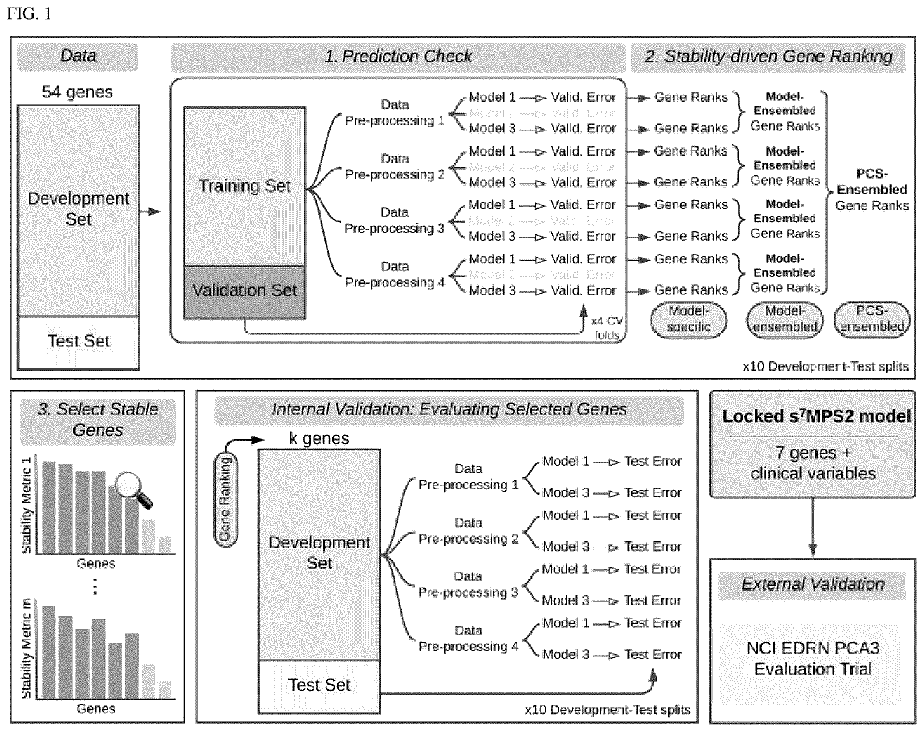

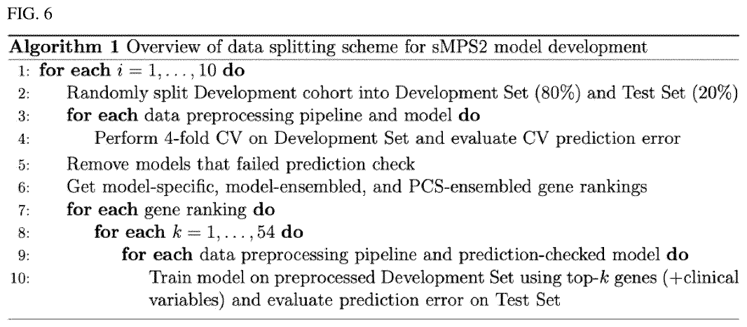

Leveraging expression data from the 54 nominated genes and available clinical variables that are generally associated with high-grade PCa (age, race, family history of prostate cancer, abnormal DRE, prior negative biopsy, and PSA) [17], the simplified MPS2 model (sMPS2) to predict high-grade PCa defined as grade group 2 or higher Pca was developed. Using the Development Cohort data, three main development stages (FIG. 1) were utilized—(1) prediction check, (2) stability-driven gene ranking, and (3) selection of stable genes—each heavily rooted in the PCS framework for veridical data science (Yu and Kumbier 2020; Yu and Barter 2024). Proper data splitting is important enable the generalizability of our developed model. The data splitting procedure in outlined in FIG. 6.

### Prediction Check

In the first stage of the model development process, a prediction check that assessed model prediction performance was used in order to filter out models which may not accurately represent the scientific phenomena under study as indicated by their poor prediction performance [6]. Specifically, the Development Cohort was randomly split into a Development (80%) and Test Set (20%). Then using the Development Set, 4-fold cross-validation (CV) was performed. For each CV fold, each model was trained on each of the four preprocessed datasets using the training fold and evaluated the prediction error on the validation fold. The covariates used included the 54 nominated genes from the original MPS2 study and freely-available clinical variables that are generally thought to be associated with high-grade PCa (age, race, family history of prostate cancer, abnormal DRE, prior negative biopsy, and PSA) [17]. The cross-validation (CV) error was computed using three different evaluation metrics—area under the receiver operating characteristic curve (AUROC), area under the precision recall curve (AUPRC), and classification accuracy—and averaged across the four CV folds. This process was repeated for 10 different Development-Test splits. Models that did not outperform ordinary logistic regression in any of the data preprocessing pipelines across the three evaluation metrics were excluded from further analysis and failed the prediction check.

### Stability-Driven Gene Ranking

For those models that passed the prediction check, a ranking of gene (or feature) importances was computed for each preprocessing-model combination. More precisely, for each data preprocessing pipeline and model specification, a measure of feature importance was computed from the model fit on each CV training fold, averaged the feature importance measures across the 4 folds, and ranked the features according to this averaged feature importance for the given method. For random forest (RF) and random forest+ (RF+), mean decrease in impurity (MDI) and mean decrease in impurity+ (MDI+) were used, respectively, as the feature importance measures. For logistic regression and logistic regression with L2 (ridge) regularization, feature importance was measured using the magnitude of the estimated regression coefficients. For logistic regression with L1 (Lasso) or the combined L1+L2 (clastic net) regularization, feature importance was measured by the number of times each feature had a non-zero coefficient across the 4 CV folds; if there are ties, these ties were broken based upon the magnitude of the estimated regression coefficients. Note that the covariate data was centered and scaled to have mean 0 and variance 1 prior to fitting these models. Additionally, while clinical variables were used as covariates in the trained models (and included in the feature importance computation), these clinical variables were dropped when computing the feature importance rankings since our aim is to identify the most important genes. Thus far, the aggregation of feature importances and resulting feature importance ranking is model-specific, i.e., each model or method yields its own feature importance ranking.

However, among the models that passed the prediction check and yielded fairly similar prediction performance, it is unclear whether or why one should trust the feature importance rankings from one method over another. Following the stability principle of the PCS framework [6], the features that were most stably important across these similarly-performing prediction models were examined by averaging the model-specific feature ranks across models, yielding a model-ensembled feature importance ranking, and across both data pre-processing pipelines and models, yielding a PCS-ensembled feature importance ranking. Note that this stability-driven feature ranking is performed per Development-Test split, using the same splits as that in the prediction check (FIG. 6).

### Selection of Stable Genes

After obtaining the various gene rankings for each Development-Test split, the stability of the top-ranked genes were examined across the different Development-Test splits via various metrics (applying the stability principle in PCS to metric choices) in order to identify the sparse set of stable genes used in the final simplified MPS2 model. The first stability metric was the proportion of times each gene was ranked in the top k across all preprocessing-model specifications and Development-Test splits (4 data preprocessing pipelines×6 models that passed the prediction check×10 splits=240 total configurations). Here, several different choices of k: k=5 and 10 were used to identify genes that were almost always the most important (˜top 10% or 20%) out of the genes under consideration, and k=17 for comparison with the original MPS2 study [5]. The second stability metric was the average PCS-ensembled feature importance ranking across all Development-Test splits. Note that this is equivalent to the average feature importance ranking across all data preprocessing pipelines, models, and Development-Test splits. The third stability metric is the standard deviation (SD) of the gene's importance rankings across all data preprocessing pipelines, models, and Development-Test splits. Using these various stability metrics, genes were identified which were frequently ranked in the top 5, 10, and 17 features, had a high average feature ranking (i.e., ranked close to 1), and whose feature rank was highly stable (or had low SD or variability) across the different preprocessing pipelines, models, and Development-Test splits. A heuristic approach, combined with expert domain knowledge, guided the final selection of genes. These selected genes were locked prior to internal and external validation, and thus, these validation studies provide a proper, unbiased assessment of the model's generalizability.

### Internal Validation for Evaluating Selected Genes

To evaluate the gene rankings from Stage 2 as well as the choice of the number of selected genes, an internal validation was performed using the test set from each of our 10 Development-Test splits (the same splits used in Stage 1). That is, for each Development-Test split, gene ranking from that given Development-Test split, and choice of k (k=1, 2, 3, 4, 5, 6, 7, 10, 12, 15, 17, 20, 25, 30, 40, 54), (a) the top k-ranked genes and the available clinical features as covariates were selected, (b) each prediction-checked model was trained on each preprocessed Development set split, and (c) the prediction error (i.e., AUROC, AUPRC, and classification accuracy) was evaluated on the test set.

These errors were then averaged across the 10 Development-Test splits. These averaged prediction errors were examined across different choices of the gene panel size k and prediction models. Different ways of obtaining the gene rankings (i.e., model-specific, model-ensembled, and PCS-ensembled) were also evaluated.

Final Simplified MPS2 Model (sMPS2)

The final covariate gene set in the simplified MPS2 model consisted of the 6 topmost important stable genes: T2: ERG, SCHLAP1, OR51E2, TFF3, PCAT14, and PCA3. Using these 6 genes and 6 clinical features (age, race, family history of prostate cancer, abnormal DRE, prior negative biopsy, and PSA) as covariates, a logistic regression was trained with L2 (ridge) regularization to predict high-grade PCa using the full Development Cohort dataset. This final trained model (estimated coefficients in Table 1) is referred to as the s7MPS2 model as it requires the measurement of 7 genes (i.e., the 6 genes used as covariates and the reference gene KLK3 which is necessary for data preprocessing). Here, a logistic regression with L2 (ridge) regularization was used due to its strong performance in the internal validation assessment. Since the inclusion of prostate volume in clinical models is well-known to improve the prediction of high-grade PCa [18,19], an analogous model, termed s7MPS2+, which includes all of the covariates in s7MPS2 plus prostate volume, for use when a patient's prostate volume is readily available, was trained. For comparison, the external validation results for the s8MPS2 model, which is analogous to s7MPS2 but includes the top 7 most stable genes as covariates (T2: ERG, SCHLAP1, OR51E2, TFF3, PCAT14, PCA3, and APOC1) was analyzed. These simplified MPS2 models were locked prior to external validation.

### Model Validation on Blinded, External Cohort

The final locked simplified MPS2 models were evaluated on two blinded, external validation cohorts. The AUROC from the locked simplified MPS2 models were compared against MPS [3] and MPS2 [5].

### Results

Development of the Simplified MyProstateScore 2.0 (sMPS2) Model

Grounded by the PCS framework for veridical data science [6], a stability-driven machine learning pipeline was used to build a robust and accurate risk score model of high-grade PCa using substantially fewer genes than MPS2. In this PCS-guided development pipeline, the accuracy and stability of modeling results was rigorously assessed across both data preprocessing and modeling pipelines in order to account for the inherent uncertainty arising from such human judgment calls and thus more faithfully capture the uncertainty and generalizability when deployed in reality [6]. Briefly, the PCS-guided development pipeline for sMPS2 consists of three main stages: (1) A prediction check stage, where the prediction performance for a variety of machine learning models was evaluated across four different data preprocessing pipelines and filtered out models with poor prediction performance; (2) A stability-driven gene ranking stage, where the importance of each gene was ranked according to both its magnitude of importance and the stability of its importances across model fits, data preprocessing pipelines, and data splits; and (3) A selection of stable genes stage, where the set of most stable important genes was selected for use in the final, locked sMPS2 model. Details regarding each step are provided above. Here, by identifying and focusing on the most important genes that were highly stable across both data preprocessing and modeling choices, it was ensured that the final locked sMPS2 model was not solely a random artifact resulting from human analysis decisions, but rather a robust clinical risk model, which was shows to have highly predictive and generalizable performance.

### Prediction Check of Machine Learning Models Via Cross Validation

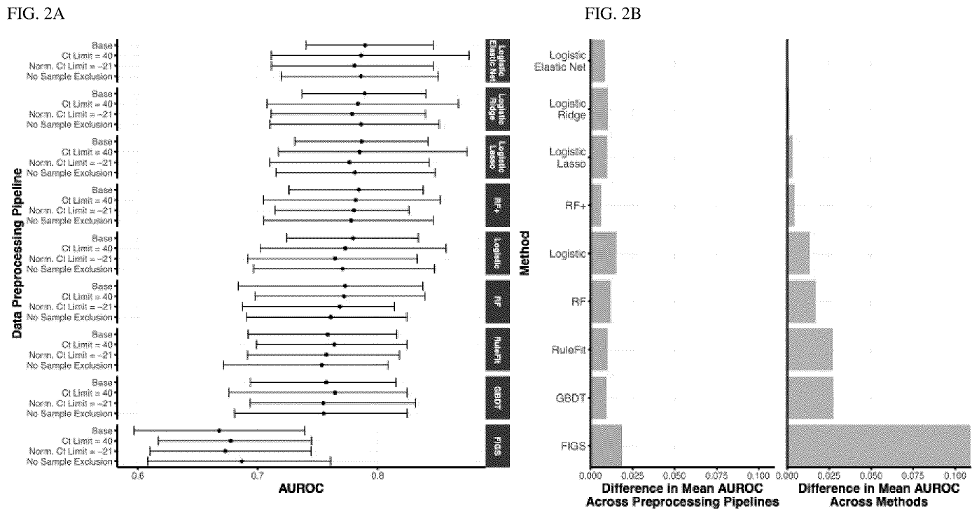

As part of the prediction check stage in the development of sMPS2 (FIG. 1), the cross-validation prediction performance for models trained with all 54 genes and available clinical variables was assessed across nine different machine learning models and four different preprocessing pipelines. Results are shown in FIG. 2 and provide the analogous AUPRC and classification accuracy results in FIG. 6. As shown in FIG. 2A, the linear-based models (i.e., both regularized and unregularized logistic regression) tended to outperform the non-linear tree-based models (RF, GBDT, RuleFit, and FIGS), indicating some smooth underlying structure in the data which can be more easily captured via linearity as opposed to trees (which are non-smooth piecewise constant functions). This is further supported by the observation that RF (AUROC 0.769) yielded a lower AUROC than RF+ (AUROC 0.781), a generalization of RF which models both smooth linear structure and nonlinear tree structure.

Notably, the variation in prediction accuracy across data preprocessing pipelines was substantially smaller than the variation in prediction accuracy across models. FIG. 2B shows the range of mean AUROCs across the four data preprocessing pipelines for each method (FIG. 2B, left), compared to the difference between each method's mean AUROC and that of the best-performing method (logistic regression with clastic net regularization) (FIG. 2B, right). Across all methods, the range of mean AUROCs across data preprocessing pipelines never exceeded 0.020 whereas the difference between logistic regression with elastic net regularization (the best-performing model) and RuleFit, GBDT, and FIGS were larger, exhibiting differences of 0.027, 0.027, and 0.109, respectively. These observations provide evidence that the trained models are not highly dependent on human choices made during the data preprocessing pipeline—a crucial stability check for fostering trust in our model development process.

Before proceeding to stage 2, this prediction check was used to filter out models with poor prediction performance, a possible indicator that the model does not accurately reflect reality and would generate unreliable interpretations [6]. Here, ordinary logistic regression was used as the “reference” model given its simplicity yet decent cross-validated prediction performance (AUROC 0.772) in this problem, and all models with worse prediction performance than logistic regression were dropped. Specifically, this prediction check excludes RuleFit, GBDT, and FIGS from the remainder of the analysis. Note that though RF (AUROC 0.769) has slightly lower prediction performance than logistic regression on average across the different data preprocessing pipelines; RF was not excluded since at least one of its data preprocessing pipelines led to higher accuracy than that for logistic regression. In other words, the uncertainty due to data preprocessing is larger than the modeling difference between RF and logistic regression. It was thus determined RF passed the prediction check and it was included in the remainder of the analysis.

Stability-Driven Genes Associated with High-Grade Prostate Cancer

Having filtered out poor-performing prediction models and established that the prediction-checked models are indeed robust to different data preprocessing choices, genes which were both ranked highly important and highly stable across the four data preprocessing pipelines, six prediction-checked models, and ten development-test splits (i.e., 4×6×10=240 combinations), were identified for use in the sMPS2 model.

### Top-Ranked Genes Across all Data Preprocessing Pipelines and Models

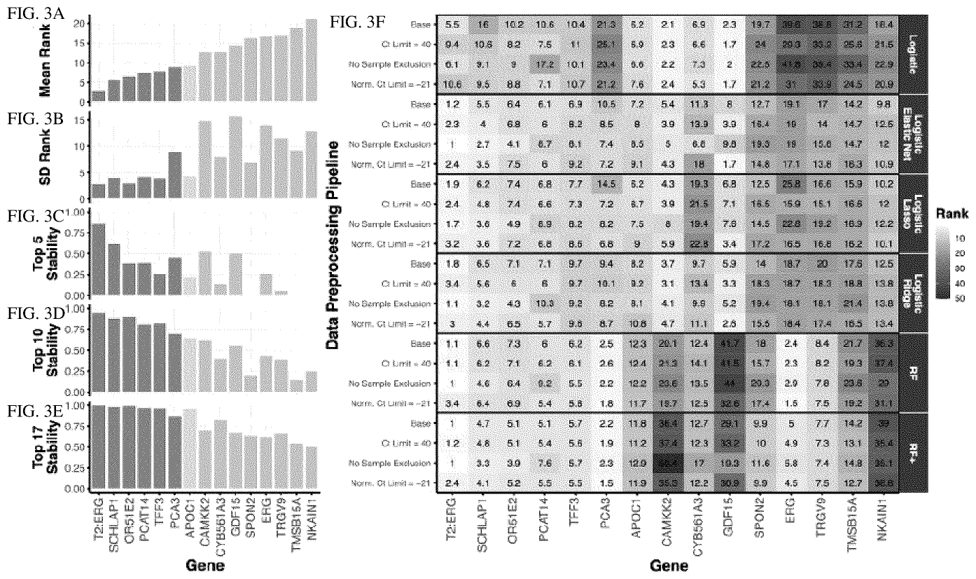

In FIG. 3A, the mean ranking of each gene across the 240 preprocessing-model-split combinations alongside numerous stability metrics is shown, including the standard deviation of each gene's ranking (FIG. 3B) and the proportion of times (out of 240) that the gene ranked in the top 5, 10, or 17 (out of 54) (FIG. 3C-E). The top six-ranked genes T2: ERG, SCHLAP1, OR51E2, TFF3, PCAT14, and PCA3 were all highly stable, each appearing in the top 10 ranked genes in more than 70% of the preprocessing-model-split combinations. The seventh-ranked gene, APOC1, also appears to be stably important; however, its stability declines when using only two of the four logistic-based regression models in the PCS-ensembled gene rankings.

When examining the gene rankings per data preprocessing pipeline and method in FIG. 3F, the robustness and stability of these trained models was confirmed across data preprocessing choices, not only in terms of their resulting prediction accuracy but also in terms of their most important genes and model architecture. FIG. 3F further reveals that the two genes T2: ERG and PCA3 comprising the original MPS model were stably ranked as the top two most important genes according to RF and RF+. Moreover, the top 6 genes were particularly stable across the regularized logistic regression models, RF, and RF+ fits while the ordinary logistic regression model generally produced a different set of gene rankings. The ordinary logistic regression model did not rank PCA3 highly and is the main source of instability seen in the high SD for PCA3 in FIG. 3B.

Top-Ranked Genes from Specific Models

Beyond these top genes, FIG. 3F illuminates several additional gene ranking patterns across the different models and data preprocessing pipelines. First, there are genes, such as CAMKK2 and GDF15, that tend to be more highly ranked when considering only linear structure (in the logistic-based models) while other genes, such as ERG and TRGV9, tend to be more highly ranked when allowing for nonlinear structures (i.e., in the tree-based models, RF and RF+). The top 6 genes (T2: ERG, SCHLAP1, OR51E2, TFF3, PCAT14, and PCA3), together with the reference gene KLK3, were selected for use in the final simplified 7-gene MPS2 model (s7MPS2), as these genes were highly stable across the various data-preprocessing, modeling, and Development-Test split combinations. Given the borderline stability status of APOC1, a simplified 8-gene MPS2 model (s8MPS2), which includes those genes in s7MPS2 along with APOC1 was also developed.

Internal Assessment and Validation of the sMPS2 Models

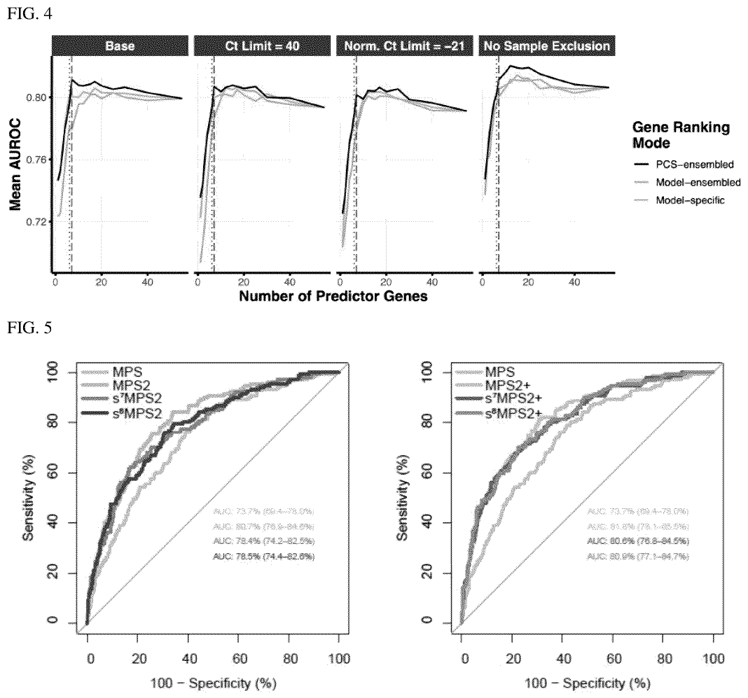

The stability of the feature rankings is the primary factor when selecting the number of top-ranked genes in our final simplified MPS2 model. To assess the impact of this choice of the gene panel size (i.e., the number of top-ranked genes used in the model) on the prediction accuracy, an internal validation assessment was performed using the test set. FIG. 4 highlights the test prediction accuracies when using the logistic regression model with ridge regularization (other model results can be found in FIG. 6). Taking the top 7 PCS-ensembled genes (as in s8MPS2) yielded the highest test AUROCs in the base and the Ct Limit=40 data preprocessing pipelines (0.811 and 0.807, respectively) while also demonstrating competitive performance in the remaining two data preprocessing pipelines. The top 6 PCS-ensembled genes (as in s7MPS2) yielded similarly strong AUROCs, showing only a 0.01 drop in AUROC compared to taking the top 7 PCS-ensembled genes across all data preprocessing pipelines.

Moreover, like the cross-validation prediction accuracies, these test prediction accuracies were very stable across the different data preprocessing choices. In particular, the AUROCs when taking the top 6 and 7 PCS-ensembled genes ranged between [0.788, 0.801] (difference of 0.013) and [0.801, 0.811] (difference of 0.010), respectively, across the different data preprocessing pipelines. When taking the top 17 genes using logistic regression with elastic net regularization as done in the original MPS2 model, the test prediction performance was also highly stable across data preprocessing pipelines, giving an AUROC range of [0.800, 0.811] (difference of 0.012). This demonstrates the robustness of both the sMPS2 models and the original MPS2 model against alternative data preprocessing choices and indicates that these potentially different, but equally-reasonable choices do not solely drive downstream conclusions.

The impact of the stability-driven PCS-ensembled ranking approaches, which averages the gene rankings across both data preprocessing pipelines and models, was compared to alternative approaches-namely, a model-ensembled approach, which averages the gene rankings across models only, and a model-specific approach, which produces a unique gene ranking per model and data preprocessing choice. Across all data preprocessing pipelines and gene panel sizes, the top genes according to the PCS-ensembled gene rankings led to higher prediction accuracies than that from the model-ensembled or the model-specific gene rankings (FIG. 4). This pattern also holds across different choices of prediction models (FIG. 6).

### External Cohort Validation

When evaluated on the NCI EDRN PCA3 Evaluation trial [7], the locked s7MPS2 model yielded an AUROC of 0.784 (95% confidence interval [CI], 0.742-0.825) for predicting high-grade PCa (FIG. 5). This was a 4.7% improvement over MPS, which gave an AUROC of 0.737 (95% CI, 0.694-0.780), and only a 2.3% drop relative to the more complex 18-gene MPS2 model, which gave an AUROC of 0.807 (95% CI, 0.769-0.846).

In the case when prostate volume is available, the s7MPS2+ model gave an AUROC of 0.806 (95% CI, 0.768-0.845), which was only a 1.2% drop relative to MPS2+ (AUROC: 0.818, 95% CI: 0.781-0.855). 7MPS2 is compared to s8MPS2 in FIG. 5, showing that the improvement when adding one additional gene (APOC1) is small. s8MPS2 and s8MPS2+ yielded AUROCs of 0.785 (95% CI, 0.744-0.826) and 0.809 (95% CI, 0.771-0.847), respectively. Importantly, the drops in AUROC (2.3%/2.2% for s7MPS2/s8MPS2 and 1.2%/0.9% for s7MPS2+/s8MPS2+ relative to MPS2 and MPS2+, respectively) are within the 1-2% uncertainty intervals induced by different data preprocessing choices. These AUROCs showcase the overall high diagnostic accuracy of the sMPS2 models.

However, from a clinical perspective, it is also important to evaluate the performance of a practical clinical testing approach using a specific decision threshold that yields high sensitivity (e.g., 95%) for high-grade PCa. At this 95% sensitivity, s7MPS2/s8MPS2 provided a specificity of 32%/30%, a negative predictive value (NPV) of 96%/96%, and positive predictive value (PPV) of 26%/26% (Table 2). This corresponds to an estimated reduction of 318/297 unnecessary biopsies avoided per 1000 patients based upon the s7MPS2/s8MPS2 models. More notably, both s7MPS2+ and s8MPS2+ achieve similar, if not higher, specificity (40.7%), NPV (96.8%), and PPV (28.9%) than MPS2+ (40.5% specificity, 96.8% NPV, 28.9% PPV) and leads to an estimated 407 unnecessary biopsies avoided per 1000 patients under this clinical testing approach at 95% sensitivity.

TABLE 1
Prediction methods, software implementations, and hyperparameters under study.

| Prediction Method | Implementation | Hyperparameters |
|---|---|---|
| Logistic regression | ‘LogisticRegression( )’ in | No hyperparameters |
| | sklearn python package | |
| Logistic regression with L₁ | ‘LogisticRegression(penalty = | C = 10ⁱ for i = −3, −2.5, 2, . . . , 2, |
| (Lasso) regularization | “l1”)’ in sklearn python | 2.5, 3 |
| | package | |
| Logistic regression with L₂ | ‘LogisticRegression(penalty = | C = 10ⁱ for i = −3, −2.5, 2, . . . , 2, |
| (ridge) regularization | “l2”)’ in sklearn python | 2.5, 3 |
| | package | |
| Logistic regression with | ‘LogisticRegression(penalty = | C = 10ⁱ for i = −3, −2.5, 2, . . . , 2, |
| combined L₁ + L₂ (elastic net) | “elasticnet”)’ in sklearn | 2.5, 3 |
| regularization | python package | l1_ratio = 0.1, 0.25, 0.5, 0.75, |
| | | 0.9 |
| Random forest | ‘RandomForestClassifier( )’ in | min_samples_leaf = 1, 3, 5, |
| | sklearn python package | 10 |
| | | n_estimators = 500 |
| Gradient boosting decision | ‘GradientBoostingClassifier( )’ | learning_rate = 0.05, 0.1. |
| trees | in skleam python package | 0.15, |
| | | min_samples_leaf = 1, 5, 10 |
| | | max_depth = 3, 5 |
| | | n_estimators = 500 |
| RuleFit | ‘RuleFitClassifier( )’ in | max_rules = 5, 10, 30, 50 |
| | imodels python package | |
| Random forest+ | ‘RandomForestPlusClassifier( | Default hyperparameter grid |
| | )’ in imodels python package | used |
| Fast interpretable greedy-tree | ‘FIGSClassifier( )’ in imodels | max_rules = 5, 10, 12, 15, 20, |
| sums | python package | 30, 50 |

TABLE 2
Performance of MPS2, MPS2+ and corresponding simplified
MPS2 models (7- and 8-biomarkers) in the validation cohort

| | | | | | Estimated |
|---|---|---|---|---|---|
| | | | | | unnecessary |
| | | | | | biopsies avoided |
| | % | | | | per 1000 |
| Model | Sensitivity | Specificity | NPV | PPV | patients |
| MPS | 95.0 | 23.0 | 94.4 | 23.9 | 230 |
| MPS2 | 95.0 | 37.0 | 96.5 | 27.7 | 370 |
| s⁷MPS2 | 95.0 | 31.8 | 95.9 | 26.1 | 318 |
| s⁸MPS2 | 95.0 | 29.7 | 95.7 | 25.6 | 297 |
| MPS2+ | 95.0 | 40.5 | 96.8 | 28.9 | 405 |
| s⁷MPS2+ | 95.0 | 40.7 | 96.8 | 28.9 | 407 |
| s⁸MPS2+ | 95.0 | 40.7 | 96.8 | 28.9 | 407 |

[1] R. Etzioni, A. Tsodikov, A. Mariotto, A. Szabo, S. Falcon, J. Wegelin, D. DiTommaso, K. Karnofski, R. Gulati, D. F. Penson and E. Feuer, Quantifying the role of PSA screening in the US prostate cancer mortality decline, Cancer Causes Control. 19 (2008), 175-181.
[2] S. A. Tomlins, D. R. Rhodes, S. Perner, S. M. Dhanasekaran, R. Mehra, X.-W. Sun, S. Varambally, X. Cao, J. Tchinda, R. Kuefer, C. Lee, J. E. Montie, R. B. Shah, K. J. Pienta, M. A. Rubin and A. M. Chinnaiyan, Recurrent fusion of TMPRSS2 and ETS transcription factor genes in prostate cancer, Science. 310 (2005), 644-648.
[3] S. A. Tomlins, S. M. J. Aubin, J. Siddiqui, R. J. Lonigro, L. Sefton-Miller, S. Miick, S. Williamsen, P. Hodge, J. Meinke, A. Blase, Y. Penabella, J. R. Day, R. Varambally, B. Han, D. Wood, L. Wang, M. G. Sanda, M. A. Rubin, D. R. Rhodes, B. Hollenbeck, K. Sakamoto, J. L. Silberstein, Y. Fradet, J. B. Amberson, S. Meyers, N. Palanisamy, H. Rittenhouse, J. T. Wei, J. Groskopf and A. M. Chinnaiyan, Urine TMPRSS2: ERG fusion transcript stratifies prostate cancer risk in men with elevated serum PSA, Sci Transl Med. 3 (2011), 94ra72.
[4] K. A. Moses, P. C. Sprenkle, C. Bahler, G. Box, S. V. Carlsson, W. J. Catalona, D. M. Dahl, M. Dall'Era, J. W. Davis, B. F. Drake, J. I. Epstein, R. B. Etzioni, T. A. Farrington, I. P. Garraway, D. Jarrard, E. Kauffman, D. Kaye, A. S. Kibel, C. A. LaGrange, P. Maroni, L. Ponsky, B. Reys, S. S. Salami, A. Sanchez, T. M. Seibert, T. M. Shaneyfelt, M. C. Smaldone, G. Sonn, M. D. Tyson, N. Vapiwala, R. Wake, S. Washington, A. Yu, B. Yuh, R. A. Berardi and D. A. Freedman-Cass, NCCN Guidelines® Insights: Prostate Cancer Early Detection, Version 1.2023, J Natl Compr Canc Netw. 21 (2023), 236-246.
[5] J. J. Tosoian, Y. Zhang, L. Xiao, C. Xie, N. L. Samora, Y. S. Niknafs, Z. Chopra, J. Siddiqui, H. Zheng, G. Herron, N. Vaishampayan, H. S. Robinson, K. Arivoli, B. J. Trock, A. E. Ross, T. M. Morgan, G. S. Palapattu, S. S. Salami, L. P. Kunju, S. A. Tomlins, L. J. Sokoll, D. W. Chan, S. Srivastava, Z. Feng, M. G. Sanda, Y. Zheng, J. T. Wei, A. M. Chinnaiyan and EDRN-PCA3 Study Group, Development and Validation of an 18-Gene Urine Test for High-Grade Prostate Cancer, JAMA Oncol. (2024).
[6] B. Yu and K. Kumbier, Veridical data science, Proc Natl Acad Sci USA. 117 (2020), 3920-3929.
[7] J. T. Wei, Z. Feng, A. W. Partin, E. Brown, I. Thompson, L. Sokoll, D. W. Chan, Y. Lotan, A. S. Kibel, J. E. Busby, M. Bidair, D. W. Lin, S. S. Taneja, R. Viterbo, A. Y. Joon, J. Dahlgren, J. Kagan, S. Srivastava and M. G. Sanda, Can urinary PCA3 supplement PSA in the early detection of prostate cancer?, J Clin Oncol. 32 (2014), 4066-4072.
[8] R. Tibshirani, Regression shrinkage and selection via the lasso, J R Stat Soc Series B StatMethodol. 58 (1996), 267-288.
[9] A. E. Hoerl and R. W. Kennard, Ridge Regression: Biased Estimation for Nonorthogonal Problems, Technometrics. 12 (1970), 55-67.
[10] H. Zou and T. Hastie, Regularization and Variable Selection Via the Elastic Net, J R Stat Soc Series B Stat Methodol. 67 (2005), 301-320.
[11] L. Breiman, Random Forests, Mach Learn. 45 (2001), 5-32.
[12] J. H. Friedman, Greedy Function Approximation: A Gradient Boosting Machine, Ann Stat. 29 (2001), 1189-1232.
[13] J. H. Friedman and B. E. Popescu, Predictive learning via rule ensembles, Aoas. 2 (2008), 916-954.
[14] A. Agarwal, A. M. Kenney, Y. S. Tan, T. M. Tang and B. Yu, MDI+: A Flexible Random Forest-Based Feature Importance Framework, arXiv [statME]. (2023).
[15] Y. S. Tan, C. Singh, K. Nasseri, A. Agarwal, J. Duncan, O. Ronen, M. Epland, A. Kornblith and B. Yu, Fast Interpretable Greedy-Tree Sums, arXiv [csLG]. (2022).
[16] D. L. Nelson, A. L. Lehninger and M. M. Cox, Lehninger Principles of Biochemistry, Macmillan, 2008.
[17] I. M. Thompson, D. P. Ankerst, C. Chi, P. J. Goodman, C. M. Tangen, M. S. Lucia, Z. Feng, H. L. Parnes and C. A. Coltman Jr, Assessing prostate cancer risk: results from the Prostate Cancer Prevention Trial, J Natl Cancer Inst. 98 (2006), 529-534.
[18] M. J. Roobol, F. H. Schröder, J. Hugosson, J. S. Jones, M. W. Kattan, E. A. Klein, F. Hamdy, D. Neal, J. Donovan, D. J. Parekh, D. Ankerst, G. Bartsch, H. Klocker, W. Horninger, A. Benchikh, G. Salama, A. Villers, S. J. Frecdland, D. M. Moreira, A. J. Vickers, H. Lilja and E. W. Steyerberg, Importance of prostate volume in the European Randomised Study of Screening for Prostate Cancer (ERSPC) risk calculators: results from the prostate biopsy collaborative group, World J Urol. 30 (2012), 149-155.
[19] D. P. Ankerst, C. Till, A. Bocck, P. Goodman, C. M. Tangen, Z. Feng, A. W. Partin, D. W. Chan, L. Sokoll, J. Kagan, J. T. Wei and I. M. Thompson, The impact of prostate volume, number of biopsy cores and American Urological Association symptom score on the sensitivity of cancer detection using the Prostate Cancer Prevention Trial risk calculator, J Urol. 190 (2013), 70-76.
[20] S. A. Tomlins, J. R. Day, R. J. Lonigro, D. H. Hovelson, J. Siddiqui, L. P. Kunju, R. L. Dunn, S. Meyer, P. Hodge, J. Groskopf, J. T. Wei and A. M. Chinnaiyan, Urine TMPRSS2: ERG Plus PCA3 for Individualized Prostate Cancer Risk Assessment, Eur Urol. 70 (2016), 45-53.
[21] J. R. Prensner, M. K. Iyer, A. Sahu, I. A. Asangani, Q. Cao, L. Patel, I. A. Vergara, E. Davicioni, N. Erho, M. Ghadessi, R. B. Jenkins, T. J. Triche, R. Malik, R. Bedenis, N. McGregor, T. Ma, W. Chen, S. Han, X. Jing, X. Cao, X. Wang, B. Chandler, W. Yan, J. Siddiqui, L. P. Kunju, S. M. Dhanasekaran, K. J. Pienta, F. Y. Feng and A. M. Chinnaiyan, The long noncoding RNA SChLAPI promotes aggressive prostate cancer and antagonizes the SWI/SNF complex, Nat Genet. 45 (2013), 1392-1398.
[22] L. L. Xu, B. G. Stackhouse, K. Florence, W. Zhang, N. Shanmugam, I. A. Sesterhenn, Z. Zou, V. Srikantan, M. Augustus, V. Roschke, K. Carter, D. G. McLeod, J. W. Moul, D. Soppett and S. Srivastava, PSGR, a novel prostate-specific gene with homology to a G protein-coupled receptor, is overexpressed in prostate cancer, Cancer Res. 60 (2000), 6568-6572.
[23] I. P. Garraway, D. Seligson, J. Said, S. Horvath and R. E. Reiter, Trefoil factor 3 is overexpressed in human prostate cancer, Prostate. 61 (2004), 209-214.
[24] S. Shukla, X. Zhang, Y. S. Niknafs, L. Xiao, R. Mehra, M. Cieślik, A. Ross, E. Schaeffer, B. Malik, S. Guo, S. M. Freier, H.-H. Bui, J. Siddiqui, X. Jing, X. Cao, S. M. Dhanasekaran, F. Y. Feng, A. M. Chinnaiyan and R. Malik, Identification and Validation of PCAT14 as Prognostic Biomarker in Prostate Cancer, Neoplasia. 18 (2016), 489-499.
[25] M. Rigau, J. Morote, M. C. Mir, C. Ballesteros, I. Ortega, A. Sanchez, E. Colás, M. Garcia, A. Ruiz, M. Abal, J. Planas, J. Reventós and A. Doll, PSGR and PCA3 as biomarkers for the detection of prostate cancer in urine, Prostate. 70 (2010), 1760-1767.
[26] E. M. Vestergaard, M. Borre, S. S. Poulsen, E. Nexø and N. Tørring, Plasma levels of trefoil factors are increased in patients with advanced prostate cancer, Clin Cancer Res. 12 (2006), 807-812.
[27] F. Y.-C. Feng, S. Zhao, J. Prensner, N. Erho, M. J. Schipper, Y. Shi, C. Magi-Galluzzi, J. Siddiqui, E. Davicioni, R. B. Den, A. Dicker, R. J. Karnes, J. T. Wei, E. A. Klein, R. B. Jenkins, A. M. Chinnaiyan and R. Mehra, Investigating the long noncoding RNA SChLAPI as a prognostic tissue and urine biomarker in prostate cancer, J Clin Orthod. 33 (2015), 7-7.
[28] S. Yang, J. Du, W. Wang, D. Zhou and X. Xi, APOC1 is a prognostic biomarker associated with M2 macrophages in ovarian cancer, BMC Cancer. 24 (2024), 364.
[29] Q. Gu, T. Zhan, X. Guan, C. Lai, N. A. Lu, G. Wang, L. Xu, X. Gao and J. Zhang, Apolipoprotein C1 promotes tumor progression in gastric cancer, Oncol Res. 31 (2023), 287-297.
[30] S. Takano, H. Yoshitomi, A. Togawa, K. Sogawa, T. Shida, F. Kimura, H. Shimizu, T. Tomonaga, F. Nomura and M. Miyazaki, Apolipoprotein C-1 maintains cell survival by preventing from apoptosis in pancreatic cancer cells, Oncogene. 27 (2008), 2810-2822.
[31] H. Zhang, Y. Wang, C. Liu, W. Li, F. Zhou, X. Wang and J. Zheng, The Apolipoprotein C1 is involved in breast cancer progression via EMT and MAPK/JNK pathway, Pathol Res Pract. 229 (2022), 153746.
W.-P. Su, L.-N. Sun, S.-L. Yang, H. Zhao, T.-Y. Zeng, W.-Z. Wu and D. Wang, Apolipoprotein C1 promotes prostate cancer cell proliferation in vitro, J Biochem Mol Toxicol. 32 (2018), c22158.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of treating prostate cancer, comprising:
a) assaying the level of expression of TMPRSS2-ERG, SCHLAP1, OR51E2, PCAT14, PCA3, and KLK4 in a sample from a subject diagnosed with prostate cancer; and
b) administering a prostate cancer treatment to a subject identified as having altered levels of expression of said genes relative to a subject without prostate cancer or a subject with low-grade prostate cancer.

2. A method of characterizing, prognosing, or recommending a treatment for prostate cancer, comprising:
a) assaying the level of expression of TMPRSS2-ERG, SCHLAP1, OR51E2, PCAT14, PCA3, and KLK4 in a sample from a subject diagnosed with prostate cancer; and
b) identifying said subject as having high-grade prostate cancer when said subject is identified as having altered levels of expression of said genes relative to a subject without prostate cancer or a subject with low-grade prostate cancer.

3. A method for informing a prostate cancer survival outcome, the method comprising:
a) detecting an amount of expression of TMPRSS2-ERG, SCHLAP1, OR51E2, PCAT14, PCA3, and KLK4, wherein the amount of expression is present in urine from a subject;
b) determining a score based on the amount of expression, wherein the score correlates with or informs the subject's likelihood of having or developing Grade Group ≥2 prostate cancer.

4. A method for identifying a subject having a high likelihood of having or developing Grade Group ≥2 prostate cancer, the method comprising detecting an amount of expression of TMPRSS2-ERG, SCHLAP1, OR51E2, PCAT14, PCA3, and KLK4, wherein the amount of expression is present in the subject's urine and indicates with a diagnostic accuracy (AUC) of ≥0.75 whether the subject has a high likelihood of having Grade Group ≥2 prostate cancer.

5. A method for identifying a likelihood of detecting Grade Group ≥2 prostate cancer from a prostate biopsy of a subject, the method comprising detecting an amount of expression of TMPRSS2-ERG, SCHLAP1, OR51E2, PCAT14, PCA3, and KLK4, wherein the amount of expression is present in the subject's urine and indicates with a diagnostic accuracy (AUC) of ≥0.75 the likelihood that Grade Group ≥2 prostate cancer would be detected from the prostate biopsy of the subject.

6. The method of any one of claims 2 to 5, further comprising administering a prostate cancer treatment to said subject.

7. The method of claim 1, wherein said subject has high-grade prostate cancer.

8. The method of claim 7, wherein said high-grade prostate cancer is Grade Group ≥2 prostate cancer.

9. The method of any of the preceding claims, further comprising detecting an amount of expression of APOC1.

10. The method of any one of the preceding claims, wherein the method further comprises determining a score based on the level or amount of expression, wherein the score indicates the subject's likelihood of having or developing Grade Group ≥2 prostate cancer.

11. The method of claim 10, further comprising generating a report comprising the score.

12. The method of claim 10 or 11, wherein Grade Group ≥2 prostate cancer is determined by a prostate biopsy of the subject.

13. The method of any one of claim 3, 10, 11 or 12, wherein the score has a diagnostic accuracy (AUC) of ≥0.75.

14. The method of any one of claims 10 to 13, further comprising forwarding the report to the subject or to a health care provider of the subject.

15. The method of any one of the preceding claims, wherein said method comprises assaying the level of expression of one to 20 additional genes.

16. The method of any one of the preceding claims, wherein the subject has not had a prior prostate biopsy.

17. The method of any one of the preceding claims, wherein the subject has had a prior negative prostate biopsy result.

18. The method of any one of the preceding claims, wherein one or more clinical variables are associated with the subject, and the method further comprises identifying at least one of the one or more clinical variables and determining the score based on the at least one of the one or more clinical variables.

19. The method of claim 18, wherein at least one of the one or more clinical variables is the subject's age, race, family history of prostate cancer, digital rectal examination (DRE) result, prostate biopsy result, prostate specific antigen (PSA) expression value based on a serum sample, multi-perimetric MRI (mpMRI) result, or any combination thereof.

20. The method of claim 19, wherein the subject's DRE or prostate biopsy is performed within 30 days before a sample of urine is obtained.

21. The method of any one of the preceding claims, wherein said prostate cancer treatment is one or more of surgery, radiation therapy, hormonal therapy, targeted therapy, chemotherapy, immunotherapy, radiopharmaceuticals, and bone-modifying drugs.

22. The method of any one of the preceding claims, wherein said level or amount of expression is the amount of mRNA or protein expressed by said genes.

23. The method of any one of the preceding claims, wherein said sample is selected from tissue, blood, plasma, serum, urine, prostate secretions, and prostate cancer cells.

24. The method of claim 23, wherein the sample is urine, and the urine is obtained within 30 minutes of the subject's DRE.

25. The method of any one of the preceding claims, further comprising determining the subject's prostate volume and determining the score based on the subject's prostate volume.

26. The method of any one of the preceding claims, wherein detecting the level or amount of expression of said genes comprises detecting an amount of mRNA expression of the genes.

27. The method of claim 26, wherein detecting the level or amount of mRNA expression comprises allowing the sample or the urine to react with a reagent composition comprising a polynucleotide reagent.

28. The method of claim 26, wherein detecting the level or amount of mRNA expression comprises synthesizing cDNA complementary to mRNA expressed by said genes, amplifying the cDNA, and detecting the cDNA.

29. The method of any one of the preceding claims, wherein the level or amount of expression of said genes is different than an amount of expression of the genes of a subject at risk for having or developing a Grade Group <2 prostate cancer or of a subject having no prostate cancer.

30. A method for screening for an amount of expression of a set of genes, comprising:
a) allowing a sample of urine from a human subject to react with a reagent for detecting an amount of expression of TMPRSS2-ERG, SCHLAP1, OR51E2, PCAT14, PCA3, and KLK4; and
b) detecting the amount of expression of the genes, wherein the amount of expression is present in the sample and the detecting comprises using an in vitro assay.

31. The method of claim 30, wherein the in vitro assay is a nucleic acid amplification assay.

32. The method of claim 31, wherein the nucleic acid amplification assay comprises performing a reverse transcription polymerase chain reaction.

33. A method for detecting an amount of mRNA expressed by a set of genes, comprising:
a) synthesizing cDNA from mRNA that is expressed by the genes and present in a sample of urine from a human subject, wherein the genes are TMPRSS2-ERG, SCHLAP1, OR51E2, PCAT14, PCA3, and KLK4;
b) amplifying the cDNA to provide amplified cDNA; and
c) detecting the amplified cDNA, wherein the amplified cDNA indicates the amount of mRNA expressed by the genes.

34. A method for detecting an amount of mRNA expressed by a set of genes, comprising:
a) isolating nucleic acid from a first composition comprising urine from a human subject to provide isolated nucleic acid;
b) allowing the isolated nucleic acid to react with a second composition comprising a reagent for detecting the amount of mRNA that is present in the first composition and expressed by TMPRSS2-ERG, SCHLAP1, OR51E2, PCAT14, PCA3, and KLK4; and
c) detecting the amount of mRNA expressed by the genes.

35. The method of any one of the preceding claims, further comprising informing the subject or a healthcare provider of the subject of treatment options for Grade Group ≥2 prostate cancer.

36. The method of any one of the preceding claims, further comprising providing to the subject or a health care provider of the subject instructions for administering to the subject a treatment for Grade Group ≥2 prostate cancer.

37. A kit comprising:
a container, the container containing a reagent composition for detecting an amount of expression of a set of genes; and
instructions for detecting the amount of expression, where the amount of expression is present in a subject's urine and the set of genes are TMPRSS2-ERG, SCHLAP1, OR51E2, PCAT14, PCA3, and KLK4.

38. The kit of claim 37, wherein the reagent composition comprises a polynucleotide reagent for detecting the amount of mRNA expressed by the set of genes.

39. The kit of claim 37 or 38, wherein the instructions are additionally for generating a report comprising a score determined by the amount of expression of the set of genes, wherein the score indicates the subject's likelihood of having or developing Grade Group ≥2 prostate cancer.

40. The kit of claim 39, wherein Grade Group ≥2 prostate cancer is determined by a prostate biopsy of the subject.

41. The kit of any one of claims 37-40, wherein the subject has not had a prior prostate biopsy.

42. The kit of any one of claims 37-41, wherein the subject has had a prior negative prostate biopsy result.

43. The kit of any one of claims 37-42, wherein one or more clinical variables are associated with the subject, and the instructions are additionally for determining the score based on at least one of the one or more clinical variables.

44. The kit of claim 42, wherein the at least one of the one or more clinical variables is the subject's age, race, family history of prostate cancer, digital rectal examination (DRE) result, prostate biopsy result, prostate specific antigen (PSA) expression value based on a serum sample, multi-perimetric MRI (mpMRI) result, or any combination thereof.

45. The kit of any one of claims 37-43, wherein the instructions are additionally for determining the score based on the subject's prostate volume.

46. The kit of any one of claims 37-44, wherein the instructions are additionally for informing the subject of treatment options for Grade Group ≥2 prostate cancer.

47. The kit of any one of claims 37-45, wherein the report comprises treatment options for Grade Group ≥2 prostate cancer.

48. The kit of any one of claims 37-46, wherein the instructions are additionally for administering to the subject a treatment for Grade Group ≥2 prostate cancer.

49. The method of any one of claims 1-38, wherein the method does not comprise performing a prostate biopsy on the subject.

50. The method of any one of claims 1-38, wherein the method further comprises performing a prostate biopsy on the subject.

51. The method of any one of claims 1-38, wherein the method further comprises recommending to the subject or the subject's health care provider that the subject undergo a prostate biopsy.

52. The method of claim 49 or 50, wherein the prostate biopsy indicates the subject has Grade Group ≥2 prostate cancer.

53. The method of claim 49 or 50, wherein the prostate biopsy indicates the subject does not have Grade Group ≥2 prostate cancer.


SEQUENCE LISTING (7 sequences)

SEQ ID NO: 1 - DNA, 293 bp, synthetic construct
  source 1..293: mol_type = other DNA
taggcgcgagctaagcaggaggcggaggcggaggcggagggcgaggggcggggagcgccgcctggagcgcggcaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaag

SEQ ID NO: 2 - DNA, 1574 bp, synthetic construct
  source 1..1574: mol_type = other DNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagtttcttctacgccaggtgtgtgcttagctccatgacaaaaggtgacagcttattctgcagcacacacacatcatcaaagtgggaggtggtgagactggcacactgacagtctgtcctagcagatttcagctcacactgcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 3 - DNA, 2792 bp, synthetic construct
  source 1..2792: mol_type = other DNA
cttctgggaatctccacaccctgaagacacagtgagttagcaccaccaccaggaattggcctttcagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagtggtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaactttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacagagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacttgagtataagataaaataaaattttattttaaatttt

SEQ ID NO: 4 - DNA, 514 bp, synthetic construct
  source 1..514: mol_type = other DNA
aggcggtcaggggaaggctcaggaggagggagatcaacatcaacctgccccgccccctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaa

SEQ ID NO: 5 - DNA, 1201 bp, synthetic construct
  source 1..1201: mol_type = other DNA
gagatacggcctcgtgggaagggaaagacctgaccgtcccccagcccgacacccgtaaagggtctgtgctgaggaggattagtaaaaggggaaggcctcttgcagttgagataagaggaaggcctccgtctcctgcatgtccttgggaatggaatgtcttggtgtaaaacccgatagtacattccttctattctgagagaagaaaaccaccctgtggctggagggtgaaggtactctacagtgtggtcattgaggacaagttgacgagagagtcccaagtacgtccacggtcagccttgcgacatttaaagttctacaatgaactcactggagatgcaaagaaaagtgtggagatggagacaccccaatcgactcgccagtctacaggtgtatccagcagctccaaagagacagcaaccagcaagaatgggccatagtgacgatggtggttttgtcaaaaagaaaagggggggatatgtaaggaaaagagagatcagactttcactgtgtctatgtagaaaaggaagacataagaaactccattttgatctgtactaagaaaaattgttttgccttgagatgctgttaatctgtaactttagccccaaccctgtgctcacggaaacatgtgctgtaaggtttaagggatctagggctgtgcaggatgtaccttgttaacaatatgtttgcaggcagtatgtttggtaaaagtcatcgccattctccattctcgattaaccaggggctcaatgcactgtggaaagccacaggaacctctgcccaagaaagcctggctgttgtgggaagtcagggaccccgaatggagggaccagctggtgctgcatcaggaaacataaattgtgaagatttcttggacatttatcagtttccaaaattaatacttttataatttcttacacctgtcttactttaatctcttaatcctgttatctttgtaagctgaggatatacgtcacctcaggaccactattgtacaaattgattgtaaaacatgttcacatgtgtttgaacaatatgaaatcagtgcaccttgaaaatgaacagaataacagtgattttagggaacaaaggaagacaaccataaggtctgactgcctgaggggtcgggcaaaaagccatatttttcttcttgcagagagcctataaatggacgtgcaagtaggagagatattgctaaatt

SEQ ID NO: 6 - DNA, 3757 bp, synthetic construct
  source 1..3757: mol_type = other DNA
acagaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 7 - DNA, 1416 bp, synthetic construct
  source 1..1416: mol_type = other DNA
aggcagcaggctggagctcagcccagcagtggaatccaggagcccagaggtggccgggtgctgacgtgatggccacagcaggaaatccctggggctggttcctggggtacctcatccttggtgtcgcaggatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgttcccagcccctcctccctcaggcccaggagtccaggcccccagcccctcctccctcaaaccaagggtacagatccccagcccctcctccctcagacccaggagtccagaccccccagcccctcctccctcagacccaggagtccagcccctcctccctcagacccaggagtccagaccccccagcccctcctccctcagacccaggagtccagcccctcctccctcagacccaggagtccagaccccccagcccctcctccctcagacccaggggtccaggcccccaacccctcctccctcagactcagaggtccaggcccccaacccctccttccccagacccagaggtccaggtcccagcccctcctccctcagacccagcggtccaatgccacctagactctccctgtacacagtgcccccttgtggcacgttgacccaaccttaccagttggtttttcattttttgtccctttcccctagatccagaaataaagtctaagagaagcgca